SEQUENCE LISTING (137 sequences)

SEQ ID NO: 1 - protein, 10 aa, Mouse
GFNIKDFYMH

SEQ ID NO: 2 - protein, 17 aa, Mouse
WIDPENGDSGYAPKFQG

SEQ ID NO: 3 - protein, 8 aa, Mouse
YYGDYEGY

SEQ ID NO: 4 - protein, 10 aa, Mouse
SASSSVSYMH

SEQ ID NO: 5 - protein, 7 aa, Mouse
STSNLAS

SEQ ID NO: 6 - protein, 9 aa, Mouse
QQRSSYPFT

SEQ ID NO: 7 - protein, 117 aa, Mouse
QVKLQQSGAELVGSGASVKLSCTTSGFNIKDFYMHWVKQRPEQGLEWIGWIDPENGDSGYAPKFQGKATMTADSSSNTAYLQLSSLTSEDTAVYYCNAYYGDYEGYWGQGTTVTVSS

SEQ ID NO: 8 - protein, 108 aa, Mouse
DIELTQSPAIMSASPGEKVTITCSASSSVSYMHWFQQKPGTSPKLWIYSTSNLASGVPARFSGSGSGTSYSLTISRMEAEDAATYYCQQRSSYPFTFGSGTKLEIKRA

SEQ ID NO: 9 - DNA, 30 bp, Mouse
ggcttcaacattaaagacttctatatgcac

SEQ ID NO: 10 - DNA, 51 bp, Mouse
tggattgatcctgagaatggtgattctggttatgccccgaagttccagggc

SEQ ID NO: 11 - DNA, 24 bp, Mouse
tactatggtgactacgaaggctac

SEQ ID NO: 12 - DNA, 30 bp, Mouse
agtgccagctcaagtgtaagttacatgcac

SEQ ID NO: 13 - DNA, 21 bp, Mouse
agcacatccaacctggcttct

SEQ ID NO: 14 - DNA, 27 bp, Mouse
cagcaaaggagtagttacccattcacg

SEQ ID NO: 15 - DNA, 351 bp, Mouse
caggtcaagctgcagcagtctggggcagagcttgtggggtcaggggcctcagtcaaattgtcctgcacaacttctggcttcaacattaaagacttctatatgcactgggtgaagcagaggcctgaacagggcctggagtggattggatggattgatcctgagaatggtgattctggttatgccccgaagttccagggcaaggccaccatgactgcagactcatcctccaacacagcctacctgcagctcagcagcctgacatctgaggacactgccgtctattactgtaatgcatactatggtgactacgaaggctactggggccaagggaccacggtcaccgtctcctca

SEQ ID NO: 16 - DNA, 324 bp, Mouse
gacatcgagctcactcagtctccagcaatcatgtctgcatctccaggggagaaggtcaccataacctgcagtgccagctcaagtgtaagttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagtagttacccattcacgttcggctcggggaccaagctggaaataaaacgggcg

SEQ ID NO: 17 - protein, 15 aa, Mouse
GGGGSGGGGSGGGGS

SEQ ID NO: 18 - DNA, 45 bp, Mouse
ggtggaggcggttcaggcggaggtggctctggcggtggcggatcg

SEQ ID NO: 19 - protein, 10 aa, Mouse
GGGGSGGGGS

SEQ ID NO: 20 - DNA, 15 bp, Mouse
ggtggaggcggttca

SEQ ID NO: 21 - protein, 17 aa, Mouse
WIDPENGDSDYAPKFQG

SEQ ID NO: 22 - protein, 117 aa, Mouse
QVKLQQSGAELVGSGASVKLSCTTSGFNIKDFYMHWVKQRPEQGLEWIGWIDPENGDSDYAPKFQGKATMTADSSSNTAYLQLSSLTSEDTAVYYCNAYYGDYEGYWGQGTTVTVSS

SEQ ID NO: 23 - protein, 106 aa, Mouse
DIELTQSPAIMSASPGEKVTITCSASSSVSYMHWFQQKPGTSPKLWIYSTSNLASGVPARFSGSGSGTSYSLTISRMEAEDAATYYCQQRSSYPFTFGSGTKLEIK

SEQ ID NO: 24 - DNA, 51 bp, Mouse
tggattgatcctgagaatggtgattctgattatgccccgaagttccagggc

SEQ ID NO: 25 - DNA, 351 bp, Mouse
caggtcaagctgcagcagtctggggcagagcttgtggggtcaggggcctcagtcaaattgtcctgcacaacttctggcttcaacattaaagacttctatatgcactgggtgaagcagaggcctgaacagggcctggagtggattggatggattgatcctgagaatggtgattctgattatgccccgaagttccagggcaaggccaccatgactgcagactcatcctccaacacagcctacctgcagctcagcagcctgacatctgaggacactgccgtctattactgtaatgcatactatggtgactacgaaggctactggggccaagggaccacggtcaccgtctcctca

SEQ ID NO: 26 - DNA, 318 bp, Mouse
gacatcgagctcactcagtctccagcaatcatgtctgcatctccaggggagaaggtcaccataacctgcagtgccagctcaagtgtaagttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagtagttacccattcacgttcggctcggggaccaagctggaaataaaa

SEQ ID NO: 27 - protein, 240 aa, Mouse
QVKLQQSGAELVGSGASVKLSCTTSGFNIKDFYMHWVKQRPEQGLEWIGWIDPENGDSGYAPKFQGKATMTADSSSNTAYLQLSSLTSEDTAVYYCNAYYGDYEGYWGQGTTVTVSSGGGGSGGGGSGGGGSDIELTQSPAIMSASPGEKVTITCSASSSVSYMHWFQQKPGTSPKLWIYSTSNLASGVPARFSGSGSGTSYSLTISRMEAEDAATYYCQQRSSYPFTFGSGTKLEIKRA

SEQ ID NO: 28 - protein, 238 aa, Mouse
QVKLQQSGAELVGSGASVKLSCTTSGFNIKDFYMHWVKQRPEQGLEWIGWIDPENGDSDYAPKFQGKATMTADSSSNTAYLQLSSLTSEDTAVYYCNAYYGDYEGYWGQGTTVTVSSGGGGSGGGGSGGGGSDIELTQSPAIMSASPGEKVTITCSASSSVSYMHWFQQKPGTSPKLWIYSTSNLASGVPARFSGSGSGTSYSLTISRMEAEDAATYYCQQRSSYPFTFGSGTKLEIK

SEQ ID NO: 29 - DNA, 43 bp, Artificial Sequence
  Synthetic primer
ctagtagcaactgccaccggcgtacattcacaggtcaagctgc

SEQ ID NO: 30 - DNA, 30 bp, Artificial Sequence
  Synthetic primer
tcgaaggatcactcaccttttatttccagc

SEQ ID NO: 31 - DNA, 52 bp, Artificial Sequence
  Synthetic primer
ggtcaaaagcttatggggatggtcatgtatcatcctttttctagtagcaact

SEQ ID NO: 32 - DNA, 36 bp, Artificial Sequence
  Signal
tcgatctagaaggatccactcacgttttatttccag

SEQ ID NO: 33 - protein, 19 aa, Artificial Sequence
  leader peptide
MGWSCIILFLVATATGVHS

SEQ ID NO: 34 - DNA, 32 bp, Artificial Sequence
  Synthetic primer
tctcggccggcttaagctgcgcatgtgtgagt

SEQ ID NO: 35 - protein, 11 aa, Mouse
SGFNIKDTYIH

SEQ ID NO: 36 - protein, 17 aa, Mouse
GRIDPPNDNTKDPKFQG

SEQ ID NO: 37 - protein, 7 aa, Mouse
PPFYFDY

SEQ ID NO: 38 - protein, 11 aa, Mouse
KASQNVDTNVA

SEQ ID NO: 39 - protein, 7 aa, Mouse
SASYRYS

SEQ ID NO: 40 - protein, 9 aa, Mouse
QQYNSFPYT

SEQ ID NO: 41 - protein, 116 aa, Mouse
QVKLQQSGAELVKPGASVKLSCTASGFNIKDTYIHWVKQSPEQGLEWIGWIDPPNDNTKYDPKFQGKATITADTSSNTAYMQLRSLTSEDTAVYYCALPPFYFDYWGHGTTVTVSS

SEQ ID NO: 42 - protein, 109 aa, Mouse
DIELTQSPKFMSTSVGDRVSVTCKASQNVDTNVAWYQQKPGQSPKALIYSASYRYSGVPDRFTGSGSGTDFTLTISNVQSEDLAEYFCQQYNSFPYTFGGGTKLEIKRA

SEQ ID NO: 43 - DNA, 33 bp, Mouse
tctggcttcaacattaaagacacctatatacac

SEQ ID NO: 44 - DNA, 51 bp, Mouse
ggaaggatcgatcctccgaatgataatactaaatatgacccgaagttccag

SEQ ID NO: 45 - DNA, 21 bp, Mouse
ccacccttctactttgactac

SEQ ID NO: 46 - DNA, 33 bp, Mouse
aaggccagtcagaatgtggatactaatgtagcc

SEQ ID NO: 47 - DNA, 21 bp, Mouse
tcggcatcctaccggtacagt

SEQ ID NO: 48 - DNA, 27 bp, Mouse
cagcaatataacagctttccttacacg

SEQ ID NO: 49 - DNA, 348 bp, Mouse
caggtcaaactgcagcagtctggggcagagcttgtcaagccaggggcctcagtcaagttgtcctgcacagcttctggcttcaacattaaagacacctatatacactgggtgaagcagagccctgaacagggcctggagtggattggaaggatcgatcctccgaatgataatactaaatatgacccgaagttccagggcaaggccactataacagcagacacatcctccaatacagcctacatgcagctccgcagcctgacatctgaggacactgccgtctattactgtgccctcccaccgttctactttgactactggggccatggcaccacggtcaccgtctcctca

SEQ ID NO: 50 - DNA, 327 bp, Mouse
gacatcgagctcactcagtctccaaaattcatgtccacatcagtaggagacagggtcagcgtcacctgcaaggccagtcagaatgtggatactaatgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcagcaatataacagctttccttacacgttcggaggggggaccaagctggaaataaaacgggcg

SEQ ID NO: 51 - protein, 240 aa, Mouse
QVKLQQSGAELVKPGASVKLSCTASGFNIKDTYIHWVKQSPEQGLEWIGWIDPPNDNTKYDPKFQGKATITADTSSNTAYMQLRSLTSEDTAVYYCALPPFYFDYWGHGTTVTVSSGGGGSGGGGSGGGGSDIELTQSPKFMSTSVGDRVSVTCKASQNVDTNVAWYQQKPGQSPKALIYSASYRYSGVPDRFTGSGSGTDFTLTISNVQSEDLAEYFCQQYNSFPYTFGGGTKLEIKRA

SEQ ID NO: 52 - DNA, 720 bp, Mouse
caggtcaaactgcagcagtctggggcagagcttgtcaagccaggggcctcagtcaagttgtcctgcacagcttctggcttcaacattaaagacacctatatacactgggtgaagcagagccctgaacagggcctggagtggattggaaggatcgatcctccgaatgataatactaaatatgacccgaagttccagggcaaggccactataacagcagacacatcctccaatacagcctacatgcagctccgcagcctgacatctgaggacactgccgtctattactgtgccctcccaccgttctactttgactactggggccatggcaccacggtcaccgtctcctcaggtggaggcggttcaggcggagggggctctggcggtggcggatcggacatcgagctcactcagtctccaaaattcatgtccacatcagtaggagacagggtcagcgtcacctgcaaggccagtcagaatgtggatactaatgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcagcaatataacagctttccttacacgttcggaggggggaccaagctggaaataaaacgggcg

SEQ ID NO: 53 - protein, 11 aa, Human
RASQSVSSYLA

SEQ ID NO: 54 - protein, 7 aa, Human
DSSNRAT

SEQ ID NO: 55 - protein, 9 aa, Human
LQHNTFPPT

SEQ ID NO: 56 - protein, 11 aa, Human
RASQGISSRLA

SEQ ID NO: 57 - protein, 7 aa, Human
AASSLQT

SEQ ID NO: 58 - protein, 9 aa, Human
QQANRFPPT

SEQ ID NO: 59 - protein, 14 aa, Human
AGTTTDLTYYDLVS

SEQ ID NO: 60 - protein, 7 aa, Human
DGNKRPS

SEQ ID NO: 61 - protein, 10 aa, Human
NSYVSSRFYV

SEQ ID NO: 62 - protein, 13 aa, Human
SGSTSNIGTNTAN

SEQ ID NO: 63 - protein, 7 aa, Human
NNNQRPS

SEQ ID NO: 64 - protein, 12 aa, uman
AAWDDSLNGHWV

SEQ ID NO: 65 - protein, 10 aa, Human
GFTFSSYSMN

SEQ ID NO: 66 - protein, 17 aa, Human
SISSSSSYIYYADSVKG

SEQ ID NO: 67 - protein, 7 aa, Human
VTDAFDI

SEQ ID NO: 68 - protein, 10 aa, Human
GGTFSSYAIS

SEQ ID NO: 69 - protein, 18 aa, Human
GGIIPIFGTANYAQKFQG

SEQ ID NO: 70 - protein, 16 aa, Human
GYDYYDSSGVASPFDY

SEQ ID NO: 71 - DNA, 375 bp, Human
gaggtccagctggtgcagtctggggctgaggtgaagaagcctggggcctcagtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacttttaccgcggacaaatccacgagtacagcctatatggagttgaggagcctgagatctgacgacacggccgtgtattactgtgcgagaggatacgattactatgatagtagtggcgtggcttccccctttgactactggggccagggaaccctggtcaccgtctcaagc

SEQ ID NO: 72 - protein, 125 aa, Human
EVQLVQSGAEVKKPGASVKVSCKASGGTFSSYAISWVRQAPGQGLEWMGGIIPIFGTANYAQKFQGRVTFTADKSTSTAYMELRSLRSDDTAVYYCARGYDYYDSSGVASPFDYWGQGTLVTVSS

SEQ ID NO: 73 - DNA, 333 bp, Human
cagtctgtgctgactcagccaccctcagcgtctgggacccccgggcagagggtcaccatctcttgttctggaagcacctccaacatcggtactaatactgcaaactggttccagcagctcccaggaacggcccccaaactcctcatccacaataataatcagcggccctcaggggtccctgaccgattctctggctccaagtctggcacctcagcctccctggccatcagtgggctccagtctgaggatgaggctgattattactgtgcagcatgggatgacagcctgaatggccattgggtgttcggcggagggaccaagctgaccgtcctg

SEQ ID NO: 74 - protein, 111 aa, Human
QSVLTQPPSASGTPGQRVTISCSGSTSNIGTNTANWFQQLPGTAPKLLIHNNNQRPSGVPDRFSGSKSGTSASLAISGLQSEDEADYYCAAWDDSLNGHWVFGGGTKLTVL

SEQ ID NO: 75 - DNA, 348 bp, Human
gaggtgcagctggtgcagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagtcacagatgcttttgatatctggggccaagggacaatggtcaccgtctcaagc

SEQ ID NO: 76 - protein, 116 aa, Human [sequence omitted]

SEQ ID NO: 77 - DNA, 321 bp, Human
gaaattgtgatgacacagtctccagccaccctgtctttgtctccaggggaaagagccaccctctcctgcagggccagtcagagtgttagcagctacttagcctggtaccaacagaaacctggccaggctcccaggctcctcatctatgattcatccaacagggccactggcatcccagccagattcagtggcagtgggtctgggacagacttcactctcaccatcagcagcctagagcctgaagattttgcaacttattactgtctacagcataacacttttcctccgacgttcggccaagggaccaaggtggaaatcaaa

SEQ ID NO: 78 - protein, 107 aa, Human
EIVMTQSPATLSLSPGERATLSCRASQSVSSYLAWYQQKPGQAPRLLIYDSSNRATGIPARFSGSGSGTDFTLTISSLEPEDFATYYCLQHNTFPPTFGQGTKVEIK

SEQ ID NO: 79 - DNA, 348 bp, Human
gaggtccagctggtgcagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagtcacagatgcttttgatatctggggccaagggacaatggtcaccgtctcaagc

SEQ ID NO: 80 - DNA, 330 bp, Human
cagtctgccctgactcagcctgcctccctgtctgggtctcctggacagtcgatcaccatctcctgcgctggaaccaccactgatcttacatattatgaccttgtctcctggtaccaacagcacccaggccaagcacccaaactcgtgatttatgacggcaataagcggccctcaggagtttctaatcgcttctctggctccaagtctggcaacacggcctccctgacaatctctggactccaggctgaggacgaggctgattattactgcaactcatatgtaagcagcaggttttatgtcttcggaactgggaccaaggtcaccgtccta

SEQ ID NO: 81 - protein, 110 aa, Human
QSALTQPASLSGSPGQSITISCAGTTTDLTYYDLVSWYQQHPGQAPKLVIYDGNKRPSGVSNRFSGSKSGNTASLTISGLQAEDEADYYCNSYVSSRFYVFGTGTKVTVL

SEQ ID NO: 82 - DNA, 348 bp, Human
gaagtgcagctggtgcagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaaggactcactgtatctgcaaatgaacagcctgagagccgaggacacggctgtgtattactgtgcgagagtcacagatgcttttgatatctggggccaagggacaatggtcaccgtctcaagc

SEQ ID NO: 83 - protein, 116 aa, Human
EVQLVQSGGGLVKPGGSLRLSCAASGFTFSSYSMNWVRQAPGKGLEWVSSISSSSSYIYYADSVKGRFTISRDNAKDSLYLQMNSLRAEDTAVYYCARVTDAFDIWGQGTMVTVSS

SEQ ID NO: 84 - DNA, 321 bp, Human
gacatccagttgacccagtctccatcttctgtgtctgcatctgtaggagacagagtcaccatcacttgtcgggcgagtcagggtattagtagtcggttagcctggtatcagcagaaaccagggaaagcccctaagctcctgatctatgctgcatccagtttgcaaactggggtcccatcaaggttcagcggcagtggatctgggacagatttcactctcactatcagcagcctgcagcctgaagattttgcaacttactattgtcaacaggctaacaggttccctccgactttcggccctgggaccaaagtggatatcaaa

SEQ ID NO: 85 - protein, 107 aa, Human
DIQLTQSPSSVSASVGDRVTITCRASQGISSRLAWYQQKPGKAPKLLIYAASSLQTGVPSRFSGSGSGTDFTLTISSLQPEDFATYYCQQANRFPPTFGPGTKVDIK

SEQ ID NO: 86 - DNA, 333 bp, Human
cagtctgtcgtgacgcagccgccctcagtgtctggggccccagggcagagggtcaccatctcctgcactgggagccactccaacttcggggcaggaactgatgtacattggtaccaacaccttccaggaacagcccccagactcctcattcatggagacagtaatcggccctccggggtccctgaccgattctctggctccaggtctggcacctcagcctccctggccatcactgggctccgggttgaggatgaggctgattattactgtcagtcgtatgactatggcctgagaggttgggtgttcggcggcgggaccaagctgaccgtcctt

SEQ ID NO: 87 - protein, 111 aa, Human
QSVVTQPPSVSGAPGQRVTISCTGSHSNFGAGTDVHWYQHLPGTAPRLLIHGDSNRPSGVPDRFSGSRSGTSASLAITGLRVEDEADYYCQSYDYGLRGWVFGGGTKLTVL

SEQ ID NO: 88 - DNA, 321 bp, Human
gatgttgtgatgactcagtctccatcgtccctgtctgcatctgtaggggacagagtcaccatcacttgccgggcaagtcagaacattaacaactatttaaattggtatcaacagaaaccaggaaaagcccctaagctcctgatctatgctgcctccactttgcaaagtggggtcccatcaaggttcagtggcagtggatctgggacagatttcactctcaccatcaccagcctacagcctgaagattctgcaacttattactgccaacagtattcccgttatcctcccactttcggcggagggaccaaggtggagatcaca

SEQ ID NO: 89 - protein, 107 aa, Human
DVVMTQSPSSLSASVGDRVTITCRASQNINNYLNWYQQKPGKAPKLLIYAASTLQSGVPSRFSGSGSGTDFTLTITSLQPEDSATYYCQQYSRYPPTFGGGTKVEIT

SEQ ID NO: 90 - DNA, 330 bp, Human
cagtctgccctgactcagcctgcctccgtgtctgggtctcgtggacagtcgatcaccctctcctgcaccggctccagcactgatgtgggtaattataactatatctcctggtaccaacaacacccaggccaagcccccaaactcttgatttacgatgtcactagtcggccctcaggtgtttctgatcgcttctctggctccaagtcaggcctcacggcctccctgaccatctctggactccagcctgaagacgaggctgactattactgcaactcctattctgccaccgacactcttgtttttggcggagggaccaagctgaccgtccta

SEQ ID NO: 91 - protein, 110 aa, Human
QSALTQPASVSGSRGQSITLSCTGSSTDVGNYNYISWYQQHPGQAPKLLIYDVTSRPSGVSDRFSGSKSGLTASLTISGLQPEDEADYYCNSYSATDTLVFGGGTKLTVL

SEQ ID NO: 92 - DNA, 333 bp, Human
caggctgtgctgactcagccgtcctcagtgtctggggccccaggacagagggtcaccatctcctgcactgggcaaagctccaatatcggggcagattatgatgtacattggtaccagcaatttccaggaacagcccccaaactcctcatctatggtcacaacaatcggccctcaggggtccctgaccgattctctggctccaagtctggcacctcagtctccctggtcatcagtgggctccaggctgaggatgaggctgattattattgccagtcctatgacagcagtctaagtggtttggtattcggcggagggaccaaggtgaccgtccta

SEQ ID NO: 93 - protein, 111 aa, Human
QAVLTQPSSVSGAPGQRVTISCTGQSSNIGADYDVHWYQQFPGTAPKLLIYGHNNRPSGVPDRFSGSKSGTSVSLVISGLQAEDEADYYCQSYDSSLSGLVFGGGTKVTVL

SEQ ID NO: 94 - DNA, 321 bp, Human
gacatccagttgacccagtctccatcttctgtgtctgcatctgttggagacagcgtcaccatcacttgtcgggcgagtcaggatattagcagctggttagcctggtatcaacagaaaccaggggaggcccctaagctcctgatctatgctgcatcccttcttcaaagtggggtcccatcacggttcagcggcagtggatctgggacagatttcgctctcactatcaacagcctgcagcctgaagattttgcaacttacttttgtcaacaggctgacagtttccctcccaccttcggccaagggacacggctggagattaaa

SEQ ID NO: 95 - protein, 107 aa, Human
DIQLTQSPSSVSASVGDSVTITCRASQDISSWLAWYQQKPGEAPKLLIYAASLLQSGVPSRFSGSGSGTDFALTINSLQPEDFATYFCQQADSFPPTFGQGTRLEIK

SEQ ID NO: 96 - DNA, 321 bp, Human
gacatcgagttgacccagtctccatcttccgtgtctgcatctgtgggagacagagtcaccctcacttgtcgggcgagtcagagtattaagaggtggttagcctggtatcagcagaaaccagggaaggcccctaggctcctcatctatgctgcatccactttgcaaagtggggtcccatcaaggttcagcggcggtggatctgggacagatttcactctcaccatcaacagcctgcagcctgaagattttgcaatttactactgtcaacaggctaacagtttccctcccactttcggccctgggaccaaagtggatatcaaa

SEQ ID NO: 97 - protein, 107 aa, Human
DIELTQSPSSVSASVGDRVTLTCRASQSIKRWLAWYQQKPGKAPRLLIYAASTLQSGVPSRFSGGGSGTDFTLTINSLQPEDFAIYYCQQANSFPPTFGPGTKVDIK

SEQ ID NO: 98 - DNA, 333 bp, Human
cagtctgtcgtgacgcagccgccctcagtgtctggggccccagggcagagggtcaccatctcctgcagtgggagcaggtccaacatcggggcacactatgaagtccagtggtaccagcagtttccgggagcagcccccaaactcctcatctatggtgacaccaatcggccctcaggggtccctgaccgattctctgcctcccactctggcacctcagcctcccttgccatcacagggctccaggctgaggatgaggctgattattactgccagtcgtatgacaccagtctacgtggtccggtgttcggcggagggaccaagctgaccgtccta

SEQ ID NO: 99 - protein, 111 aa, Human
QSVVTQPPSVSGAPGQRVTISCSGSRSNIGAHYEVQWYQQFPGAAPKLLIYGDTNRPSGVPDRFSASHSGTSASLAITGLQAEDEADYYCQSYDTSLRGPVFGGGTKLTVL

SEQ ID NO: 100 - DNA, 333 bp, Human
cagtctgtcgtgacgcagccgccctcagtgtctggggccccagggcagagggtcaccatctcctgcactgggagcagctccaacatcgggacaggttatgatgtacattggtaccagcaggttccaggatcagcccccaaactcctcatctatgcttacaccaatcggccctcaggggtccctgaccgattctctggctccaagtctggcatgtcagcctccctggtcatcggtggtctccaggctgaggatgaggctgattattactgccagtcctttgacgacagcctgaatggtcttgtcttcggacctgggacctcggtcaccgtcctc

SEQ ID NO: 101 - protein, 111 aa, Human
QSVVTQPPSVSGAPGQRVTISCTGSSSNIGTGYDVHWYQQVPGSAPKLLIYAYTNRPSGVPDRFSGSKSGMSASLVIGGLQAEDEADYYCQSFDDSLNGLVFGPGTSVTVL

SEQ ID NO: 102 - DNA, 333 bp, Human
cagtctgtgttgacgcagccgccctcagtgtctggggccccagggcagagggtcaccatctcctgcactgggagccactccaacttcggggcaggtactgatgtccattggtaccaacaccttccaggaacagcccccagactcctcattcatggagacactcatcggccctccggggtcgctgaccgattctctggctccaggtctggcgcctcagcctccctggccatcactgggctccgggttgaggatgaggctgattattactgtcagtcgtatgactatggcctgagaggttgggtgttcggcggcgggaccaagctgaccgtcctt

SEQ ID NO: 103 - protein, 111 aa, Human
QSVLTQPPSVSGAPGQRVTISCTGSHSNFGAGTDVHWYQHLPGTAPRLLIHGDTHRPSGVADRFSGSRSGASASLAITGLRVEDEADYYCQSYDYGLRGWVFGGGTKLTVL

SEQ ID NO: 104 - DNA, 321 bp, Human
gacatccagatgacccagtctccatcttccgtgtctgcatctataggagacagagtcaccatcacttgtcgggcgagtcagggtattgacaactggttaggctggtatcagcagaaacctgggaaagcccctaaactcctgatctacgatgcatccaatttggacacaggggtcccatcaaggttcagtggaagtggatctgggacatattttactctcaccatcagtagcctgcaagctgaagattttgcagtttatttctgtcaacaggctaaagcttttcctcccactttcggcggagggaccaaggtggacatcaaa

SEQ ID NO: 105 - protein, 107 aa, Human [sequence omitted]

SEQ ID NO: 106 - protein, 13 aa, Human
TGSHSNFGAGTDV

SEQ ID NO: 107 - protein, 7 aa, Human
GDSNRPS

SEQ ID NO: 108 - protein, 11 aa, Human
QSYDYGLRGWV

SEQ ID NO: 109 - protein, 11 aa, Human
RASQNINNYLN

SEQ ID NO: 110 - protein, 7 aa, Human
AASTLQS

SEQ ID NO: 111 - protein, 9 aa, Human
QQYSRYPPT

SEQ ID NO: 112 - protein, 14 aa, Human
TGSSTDVGNYNYIS

SEQ ID NO: 113 - protein, 7 aa, Human
DVTSRPS

SEQ ID NO: 114 - protein, 10 aa, Human
NSYSATDTLV

SEQ ID NO: 115 - protein, 14 aa, Human
TGQSSNIGADYDVH

SEQ ID NO: 116 - protein, 7 aa, Human
GHNNRPS

SEQ ID NO: 117 - protein, 11 aa, Human
QSYDSSLSGLV

SEQ ID NO: 118 - protein, 11 aa, Human
RASQDISSWLA

SEQ ID NO: 119 - protein, 7 aa, Human
AASLLQS

SEQ ID NO: 120 - protein, 9 aa, Human
QQADSFPPT

SEQ ID NO: 121 - protein, 11 aa, Human
RASQSIKRWLA

SEQ ID NO: 122 - protein, 7 aa, Human
AASTLQS

SEQ ID NO: 123 - protein, 9 aa, Human
QQANSFPPT

SEQ ID NO: 124 - protein, 14 aa, Human
SGSRSNIGAHYEVQ

SEQ ID NO: 125 - protein, 7 aa, Human
GDTNRPS

SEQ ID NO: 126 - protein, 11 aa, Human
QSYDTSLRGPV

SEQ ID NO: 127 - protein, 14 aa, Human
TGSSSNIGTGYDVH

SEQ ID NO: 128 - protein, 7 aa, Human
AYTNRPS

SEQ ID NO: 129 - protein, 11 aa, Human
QSFDDSLNGLV

SEQ ID NO: 130 - protein, 14 aa, Human
TGSHSNFGAGTDVH

SEQ ID NO: 131 - protein, 7 aa, Human
GDTHRPS

SEQ ID NO: 132 - protein, 11 aa, Human
QSYDYGLRGWV

SEQ ID NO: 133 - protein, 11 aa, Human
RASQGIDNWLG

SEQ ID NO: 134 - protein, 7 aa, Human
DASNLDT

SEQ ID NO: 135 - protein, 9 aa, Human
QQAKAFPPT

SEQ ID NO: 136 - DNA, 2351 bp, Human
ggtaccgagaaagaaccggctcccgagttctgggcatttcgcccggctcgaggtgcaggatgcagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccatgctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggaaaatcagacgacaagtattggggaaagcatcgaagtctcatgcacggcatctgggaatccccctccacagatcatgtggtataaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaa

SEQ ID NO: 137 - protein, 764 aa, Human
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSHAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLE

(19) United States
(12) Patent Application Publication
(10) Pub. No.: US 2009/0028859 A1
(43) Pub. Date: Jan. 29, 2009

(54) Bispecific antibodies that bind to vegf receptors

(76) Inventor: Zhenping Zhu, Oakland, NJ (US)

Correspondence Address: IMCLON;Lerner, David, Littenberg, Krumholz & Mentlik, LLP, 600 South Avenue West, Westfield, NJ, 07090, US

(21) Appl. No.: 10/520,026

(22) PCT Filed: Dec. 24, 2002
(86) PCT No.: PCT/US02/41372

§ 371 Date: Feb. 14, 2006

(51) Int. Cl. A61K 39/395; C07K 16/28; C12P 21/00; A61P 35/00; C12N 5/00

(52) U.S. Cl. 424/136.1; 530/387.3; 435/69.6; 435/375

(57) ABSTRACT

The present invention is directed to production of antigen-binding proteins that bind specifically to an extracellular domains of two different VEGF receptors. The bispecific antigen-binding proteins block activation of the VEGF receptors and are used to reduce or inhibit VEGF-induced cellular functions such as mitogenesis of vascular endothelial cells and migration of leukemia cells. The antigen-binding proteins of the present invention can be monovalent or multivalent, have antigen-binding sites consisting of immunoglobulin heavy chain and light chain variable domains and may further include immunoglobulin constant domains.

This application claims the benefit of U.S. Provisional Application Ser. No. 60/301,299, filed Jun. 26, 2001 and PCT/US02/20332, filed Jun. 26, 2002.

### FIELD OF THE INVENTION

The present invention is directed to production of bispecific antigen-binding proteins that bind specifically to the extracellular domains of two different VEGF receptors. The bispecific antigen-binding proteins block activation of the VEGF receptors and are used to reduce or inhibit VEGF-induced cellular functions such as initogenesis of vascular endothelial cells and migration of leukemia cells. The antigen-binding proteins of the present invention have antigen-binding sites consisting of immunoglobulin heavy chain and light chain variable domains and may be monovalent or bivalent. The antigen-binding proteins can further comprise immunoglobulin constant regions.

### BACKGROUND OF THE INVENTION

Vascular endothelial growth factors (VEGF), placenta growth factor (P1GF) and their receptors VEGPR-1/Flt-1, VEGFR-2/KDR and VEGFR-3/Flt-4 have important roles in vasculogenesis, angiogenesis and growth of tumor cells.

Vascular endothelial growth factor (VEGF) is a key regulator of vasculogenesis during embryonic development and angiogenic processes during adult life such as wound healing, diabetic retinopathy, rheumatoid arthritis, psoriasis, inflammatory disorders, tumor growth and metastasis (Ferrara, 1999, Curr. Top. Micorbiol. Immunol. 237:1-30; Klagsbrun, M. et al., 1996, Cytokine Rev. 7:259-270; Neufeld, G. et al., 1999, FASEB J. 13:9-22). VEGF is a strong inducer of vascular permeability, stimulator of endothelial cell migration and proliferation, and is an important survival factor for newly formed blood vessels. VEGF binds to and mediates its activity mainly through two tyrosine kinase receptors, VEGF receptor 1 (VEGFR-1), or fms-like tyrosine receptor 1 (Flt-1), and VEGF receptor 2 (VEGFR-2), or kinase insert domain-containing receptor (KDR; Flk-1 in mice). Numerous studies have shown that over-expression of VEGF and its receptor play an important role in tumor-associated angiogenesis, and hence in both tumor growth and metastasis (Folkman, J., 1995, Nat. Med. 1:27-31; Zhu, Z. et al., 1999, Invest. New Drugs 17:195-212). This role is further supported by studies demonstrating, for example, inhibition of tumor growth in animal models by antibodies to VEGF (Kim et al., 1993, Nature 362:841-844) and its receptors (Zhu, Z. et al., 1998, Cancer Rex. 58:3209-3214; Prewett, M. et al., 1999, Cancer Rex. 59:5209-5218).

Flt-1 and KDR have distinct functions in vascular development in embryos. Targeted deletion of genes encoding either receptor in mice is lethal to the embryo, demonstrating the physiological importance of the VEGF pathway in embryonic development. KDR-deficient mice have impaired blood island formation and lack mature endothelial cells, whereas Flt-1 null embryos fail to develop normal vasculature due to defective in the formation of vascular tubes, albeit with abundant endothelial cells. On the other hand, inactivation of Flt-1 signal transduction by truncation of the tyrosine kinase domain did not impair mouse embryonic angiogenesis and embryo development, suggesting that signaling through the Flt-1 receptor is not essential for vasculature development in the embryo. The biological responses of Flt-1 and KDR to VEGF in the adult also appear to be different. It is generally believed that KDR is the main VEGF signal transducer that results in endothelial cell proliferation, migration, differentiation, tube formation, increase of vascular permeability, and maintenance of vascular integrity. Flt-1 possesses a much weaker kinase activity, and is unable to generate a mitogenic response when stimulated by VEGF—although it binds to VEGF with an affinity that is approximately 10-fold higher than KDR. Flt-1, however, has been implicated in VEGF and placenta growth factor (PlGF)-induced migration of monocytes/macrophage and production of tissue factor.

Apart from VEGF and PlGF, several other growth factors related to VEGF have been identified: VEGF-B, VEGF-C, VEGF-D, and VEGF-E. VEGF-B, like PlGF, binds to Flt-1. VEGF-E is specific for KDR, while VEGF-C and VEGF-D can bind to KDR and another receptor, VEGFR-3 (Flt-4). In addition to their respective specific receptors, these ligands may form heterodimers that bind differentially to various receptor homo- or heterodimers and signal through different pathways.

Multispecific antibodies have been used in several small-scale clinical trials as cancer imaging and therapy agents, but broad clinical evaluation has been hampered by the lack of efficient production methods. The design of such proteins thus far has been concerned primarily with providing multi specificity. In few cases has any attention been devoted to providing other useful functions associated with natural antibody molecules.

In recent years, a variety of chemical and recombinant methods have been developed for the production of bispecific and/or multivalent antibody fragments. For review, see: Holliger, P. and Winter, G., Curr. Opin. Biotechnol. 4, 446-449 (1993); Carter, P. et al., J. Hematotherapy 4,463-470 (1995); Plückthun, A. and Pack, P., Immunotechnology 3, 83-105 (1997). Bispecificity and/or bivalency has been accomplished by fusing two scFv molecules via flexible linkers, leucine zipper motifs, C_{H}C_{L}-heterodimerization, and by association of scFv molecules to form bivalent monospecific diabodies and related structures. Multivalency has been achieved by the addition of multimerization sequences at the carboxy or amino terminus of the scFv or Fab fragments, by using for example, p53, streptavidin and helix-turn-helix motifs. For example, by dimerization via the helix-turn-helix motif of an scFv fusion protein of the form (scFv1)-hinge-helix-turn-helix-(scFv2), a tetravalent bispecific miniantibody is produced having two scFv binding sites for each of two target antigens. Improved avidity may also been obtained by providing three functional antigen binding sites. Foe example, scFv molecules with shortened linkers connecting the V_{H} and V_{L} domains associate to for a triabody (Kortt et al., 1997, Protein Eng. 10:423-433).

Production of IgG type bispecific antibodies, which resemble IgG antibodies in that they possess a more or less complete IgG constant domain structure, has been achieved by chemical cross-linking of two different IgG molecules or by co-expression of two antibodies from the same cell. One strategy developed to overcome unwanted pairings between two different sets of IgG heavy and light chains co-expressed in transfected cells is modification of the C_{H}3 domains of two heavy chains to reduce homodimerization between like antibody heavy chains. Merchant, A. M., et al., (1998) Nat. Biotechnology 16, 677-681. In that method, light chain mispairing was eliminated by requiring the use of identical light chains for each binding site of those bispecific antibodies.

In some cases, it is desirable to maintain functional or structural aspects other than antigen specificity. For example, both complement-mediated cytotoxicity (CMC) and antibody-dependent cell-mediated cytotoxicity (ADCC), which require the presence and function of Fc region heavy chain constant domains, are lost in most bispecific antibodies. Coloma and Morrison created a homogeneous population of bivalent BsAb molecules with an Fc domain by fusing a scFv to the C-terminus of a complete heavy chain. Co-expression of the fusion with an antibody light chain resulted in the production of a homogeneous population of bivalent, bispecific molecules that bind to one antigen at one end and to a second antigen at the other end (Coloma, M. J. and Morrison, S. L. (1997) Nat. Biotechnology 15, 159-163). However, this molecule had a reduced ability to activate complement and was incapable of effecting CMC. Furthermore, the C_{H}3 domain bound to high affinity Fc receptor (FcγR1) with reduced affinity. Zhu et al., PCT/USO1/16924, have described the replacement of Ig variable domains with single chain Fvs in order to produce tetrameric Ig-like proteins that (1) are bispecific and bivalent, (2) are substantially homogeneous with no constraints regarding selection of antigen-binding sites, (3) comprise Fc constant domains and retain associated functions, and (4) can be produced in mammalian or other cells without further processing. By a similar method, bispecific monovalent Fab-like proteins can be produced.

### SUMMARY OF THE INVENTION

The present invention provides antibodies that have an antigen binding site specific for a first VEEGF receptor and an antigen binding site specific for a second VEGF receptor. The antibodies are at least bivalent and may be trivalent, tetravalent or multivalent.

In a preferred embodiment, the antibody is bispecific, having one antigen binding site specific for a first VEGF receptor and a second antigen binding site specific for a second VEGF receptor. When bound to a VEGF receptor, the antibody effectively blocks interaction between the VEGF receptor and its ligand. Alternatively, or additionally, the antibody is effective to block dimerization of the VEGF receptor proteins. Compared to binding to a single VEGF receptor, dual binding can result in more potent inhibition of VEGF-stimulated cellular functions such as, for example, proliferation of endothelial cells and VEGF- and PlGF-induced migration of human leukemia cells. Antigen-binding proteins are preferably specific for mammalian VEGF receptors or more preferably for human VEGF receptors. VEGF receptors include human KDR, Flt-1 and Flt-4 and their mammalian homologs. In a particularly preferred embodiment, the antibody is specific for KDR and Flt-1.

In an embodiment of the invention, an antibody can bind specifically to an extracellular domain of a VEGF receptor and neutralizing activation of the VEGF receptor, for example, by block ligand binding or receptor dimerization. In another embodiment of the invention, a bispecific antibody can bind specifically to a VEGF receptor and inhibit angiogenesis. In yet another embodiment of the invention, an antibody can bind specifically to an extracellular domain of a VEGF receptor and reduce tumor growth.

The invention further contemplates methods of producing bispecific antigen- binding proteins that are specific for two different VEGF receptors. The antigen-binding proteins can be, for example, monovalent or bivalent. In one embodiment, diabodies are produced by coexpression and secretion of two protein chains in bacteria. A first construct encodes the V_{H} domain of a first antibody specific for the first VEGF receptor and the V_{L} domain of a second antibody specific for the second VEGF receptor. A second construct encodes the V_{L} domain of the first antibody and the V_{H} domain of the second antibody. The two chains that are expressed associate as a heterodimer with one binding site for each VEGF receptor. In another embodiment, an Ig like antibody is produced wherein a first single chain Fv (scFv) specific for a first VEGF receptor is substituted for each of the V_{H} domains and a second scFv specific for a second VEGF receptor is substituted for each of the V_{L} domains. The tetrameric antibody formed by association of two heavy and two light chains is bispecific and bivalent, and further comprises immunoglobulin constant regions.

The invention contemplates methods for neutralizing activation of a first VEGF receptor and a second VEGF receptor which comprise treating cells with a bispecific antibody of the invention. It is further contemplated to use the binding proteins in methods for inhibiting angiogenesis and reducing tumor growth.

### DESCRIPTION OF THE FIGURES

FIG. 1A is a schematic representation of the DNA constructs used for expression of scFv p1C11, scFv 6.12 and the anti-KDR x anti-Flt-1 bifunctional diabody comrising the p1C11 and Mab 6.12 antigen binding sites in E. coli.

FIG. 1B depicts expression and purification of the scFvs and the diabody. The antibodies were expressed in E.coli, purified by affinity chromatography, and analyzed by SDS-PAGE. Lane 1, scFv p1C11; lane 2, scFv 6.12; and lane 3, the bifunctional diabody. Molecular weights of markers are in kDa.

FIG. 2 demonstrates the dual specificity of the anti-KDR x anti-Fit-i bifunctional diabody. FIG. 2A shows simultaneous binding by the diabody to both KDR and Flt-1. FIGS. 2B and 2C show specific binding of the antibodies to immobilized KDR (B) and Flt-1 (C).

FIG. 3 shows inhibition of binding of KDR and Fit-1 to immobilized VEGF or PlGF by the anti-KDR x anti-Flt-1 bifunctional diabody. Various concentrations of antibodies were incubated with a fixed concentration of KDR-AP (A) or Flt-1-Fc fusion proteins (B and C) in solution at RT for 1 h, after which the mixtures were transferred to 96-well plates coated with VEGF (A and B) or PlGF (C).

FIG. 4 shows inhibition of PlGF and VEGF-induced migration of human leukemia cells by the anti-KDR x anti-Flt-1 bifunctional diabody. Panel A and D: PlGF (A) and VEGF (D) promote migration of HL60 and HEL cells in a dose-dependent manner. Panels B, C, E and F: Inhibition of PlGF (B and C), and VEGF (E and F) induced migration of human leukemia cells by the anti-KDR x anti-Flt-1 bifunctional diabody.

FIG. 5 shows inhibition of VEGF-stimulated HUVEC mitogenesis by the anti-KDR x anti-Flt-1 bifunctional diabody.

### DETAILED DESCRIPTION OF THE INVENTION

The present invention provides bispecific antibodies that are capable of binding specifically to a first VEGF receptor and to a second VEGF receptor. Of particular interest are antibodies that bind to the extracellular domains of such receptors. An extracellular domain of a VEGF receptor is herein defined includes the ligand-binding domain of the extracellular portion of the receptor, as well as extracellular portions that are involved in dimerization and overlapping epitopes. When bound to the extracellular domain of a VEGF receptor, the antibodies effectively block ligand binding and/or interfere with receptor dimerization. As a result of such binding, the antibodies neutralize activation of the VEGF receptor. Neutralizing a receptor means diminishing and/or inactivating the intrinsic ability of the receptor to transduce a signal. A reliable assay for VEGF receptor neutralization is inhibition of receptor phosphorylation. Methods of determining receptor phosphorylation are well known in the art and include, for example, measurement of phosphotyrosine with monoclonal antibodies or radioactive labels.

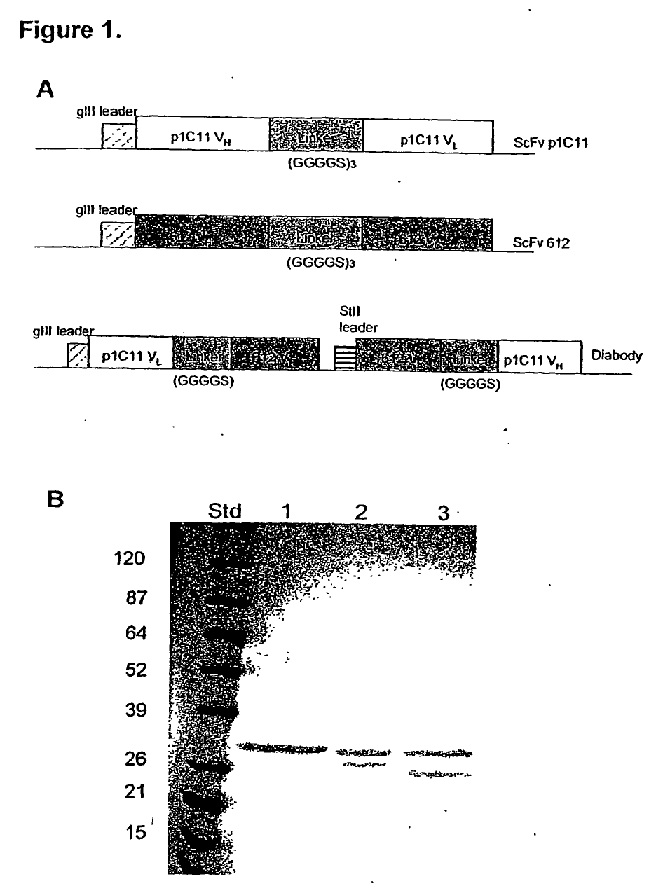

A natural antibody molecule is composed of two identical heavy chains and two identical light chains. Each light chain is covalently linked to a heavy chain by an interchain disulfide bond. The two heavy chains are further linked to one another by multiple disulfide bonds. FIG. 1 represents the structure of a typical IgG antibody. The individual chains fold into domains having similar sizes (110-125 amino acids) and structures, but different functions. The light chain comprises one variable domain (V_{L}) and one constant domain (C_{L}). The heavy chain comprises one variable domain (V_{H}) and, depending on the class or isotype of antibody, three or four constant domains (C_{H}1, C_{H}2, C_{H}3 and C_{H}4). In mice and humans, the isotypes are IgA, IgD, IgE, IgG, and IgN4, with IgA and IgG further subdivided into subclasses or subtypes. The portion of an antibody consisting of V_{L} and V_{H} domains is designated “Fv” and constitutes the antigen-binding site. A single chain Fv (scFv) is an engineered protein containing a V_{L} domain and a V_{H} domain on one polypeptide chain, wherein the N terminus of one domain and the C terminus of the other domain are joined by a flexible linker. “Fab” refers to the portion of the antibody consisting of V_{L}, V_{H}, C_{L} and C_{H}1 domains.

The variable domains show considerable amino acid sequence variablity from one antibody to the next, particularly at the location of the antigen binding site. Three regions, called “hypervariable” or “complementarity-determining regions” (CDR's) are found in each of V_{L} and V_{H}.

“Fc” is the designation for the portion of an antibody which comprises paired heavy chain constant domains. In an IgG antibody, for example, the Fc comprises C_{H}² and C_{H}³ domains. The Fc of an IgA or an IgM antibody further comprises a C_{H}4 domain. The Fc is associated with Fc receptor binding, activation of complement-mediated cytotoxicity and antibody-dependent cellular-cytoxicity. For natural antibodies such as IgA and IgM, which are complexes of multiple IgG like proteins, complex formation requires Fc constant domains.

Finally, the “hinge” region separates the Fab and Fc portions of the antibody, providing for mobility of Fabs relative to each other and relative to Fc, as well as including multiple disulfide bonds for covalent linkage of the two heavy chains.

As used herein, “antibody” refers to a binding protein that comprises antibody V_{H} and/or V_{L} domains. Antibody specificity refers to selective recognition of the antibody for a particular epitope of an antigen. Natural antibodies, for example, are monospecific. Bispecific antibodies (BsAbs) are antibodies which have two different antigen-binding specificities or sites. Where an antibody has more than one specificity, the recognized epitopes may be associated with a single antigen or with more than one antigen. Antibodies of the present invention are specific for at least a first and a second VEGF receptor, which receptors include, but are not limited to, human KDR, Flt-1, Flt-4 and their non-human homologs.

Valency refers to the number of binding sites which an antibody has for a particular epitope. For example, a natural IgG antibody is monospecific and bivalent. Where an antibody has specificity for more than one epitope, valency is calculated for each epitope. For example, an antibody which has four binding sites and recognizes a single epitope is tetravalent. An antibody with four binding sites, two binding sites having one specificity and two binding sites having a second specificity, is considered bivalent.

V_{L} and V_{H} domains for use in the present invention can be obtained, e.g., from hybridomas or phage display libraries, or from antibodies previously identified as specific for a VEGF receptor. Bispecific antibodies specific for two different receptors are exemplified, although antibodies with more than two binding sites can be engineered that are specific for more than two antigens. In one embodiment, an antibody of the invention binds to KDR and Flt-1. In another embodiment, an antibody of the invention binds to KDR and Flt-4.

An example of an antibody binding domain that binds to KDR, scFv p1C11 (SEQ ID NOS: 27, 28), was produced from a mouse scFv phage display library. (Zhu et al., 1998). p1C11 blocks VEGF-KDR interaction and inhibits VEGF-stimulated receptor phosphorylation and mitogenesis of human vascular endothelial cells (HUVEC). This scFv binds both soluble KDR and cell surface-expressed KDR on, e.g., HUVEC with high affinity (K_{d}=2.1 nM). Mab 6.12 is an example of an antibody that binds to soluble and cell surface-expressed Flt-1. A hybridoma cell line producing Mab 6.12 has been deposited as ATCC number PTA-3344 under the provisions of the Budapest Treaty on the International Recognition of the Deposit of Microorganisms for the Purposes of Patent Procedure and the regulations thereunder (Budapest Treaty).

In theory, antibodies to an individual growth factor such as VEGF would only neutralize specifically the angiogenic activity of the single ligand. In contrast, antagonistic antibodies to a VEGF receptor will not only block the angiogenic activity of VEGF, but also that of other growth factors exerting their angiogenic effects via the receptor. For example, an anti-KDR antibody will potentially block angiogenic activity of VEGF, VEGF-C, VEGF-D and VEGF-E, whereas an antibody to Flt-1 will inhibit the activity of VEGF, PlGF and VEGF-B. Furthermore, where receptor function involves dimerization, antibodies of the invention are capable of binding to one or both monomers and blocking function. For example, formation of KDR/Flt-1 heterodimers as well as KDR/KDR homodimers can be blocked by antibodies that are specific for KDR. Antibodies specific for Flt-1 can block formation of KDR/Flt-1 heterodimers and Flt-1/Flt-1 homodimers.

Antibodies of the present invention have two or more binding sites and are at least bispecific. That is, the antibodies may be bispecific even in cases where there are more than two binding sites. Antibodies of the invention include, for example, multivalent single chain antibodies, diabodies and triabodies, as well as antibodies having the constant domain structure of naturally-occurring antibodies. The antibodies can be wholly from a single species, or be chimerized or humanized. For an antibody with more than two antigen binding sites, some binding sites may be identical, so long as the protein has binding sites for two or more different antigens. That is, whereas a first binding site is specific for a first VEGF receptor, a second binding site is specific for a second, different VEGF receptor. In a preferred embodiment, the antibodies are bispecific. In a more preferred embodiment, the antibodies are designed such that a population of the antibodies is homogeneous (i.e., each and every antibody in the population has a first binding site specific for a first VEGF receptor and a second binding site specific for a second VEGF receptor).

Like natural antibodies, an antigen binding site of an antibody of the invention typically contains six complementarity determining regions (CDRs) which contribute in varying degrees to the affinity of the binding site for antigen. There are three heavy chain variable domain CDRs (CDRH1, CDRH2 and CDRH3) and three light chain variable domain CDRs (CDRL1, CDRL2 and CDRL3). The extent of CDR and framework regions (FRs) is determined by comparison to a compiled database of amino acid sequences in which those regions have been defined according to variability among the sequences.

Also included within the scope of the invention are functional antigen binding sites comprised of fewer CDRs (i.e., where binding specificity is determined by three, four or five CDRs). For example, less than a complete set of 6 CDRs may be sufficient for binding. In an embodiment of the invention, a binding protein specific for a VEGF receptor can consist of a single domain, i.e., a V_{H} or a V_{L} domain will bind specifically with high affinity.

Example of antibodies wherein binding affinity and specificity are contributed primarily by one or the other variable domain are known in the art. See, e.g., Jeffrey, P. D. et al., 1993, Proc. Nat.l. Acad. Sci. U S A 90:10310-10314, which discloses an anti-digoxin antibody which binds to digoxin primarily by the antibody heavy chain. Accordingly, single antibody domains can be identified that bind to VEGF receptors. Such single domain antibodies can be obtained, for example, from naturally occurring antibodies, or Fab or scFv phage display libraries. It is understood that, to make a single domain antibody from an antibody comprising a V_{H} and a V_{L} domain, certain amino acid substutions outside the CDR regions may be desired to enhance expression or solubility. For example, it may be necessary to modify amino acid residues that would otherwise be buried in the V_{H}-V_{L} interface.

More recently, antibodies that are homodimers of heavy chains have been discovered in camelids (camels, dromedaries and llamas). These heavy chain antibodies are devoid of light chains and the first constant domain. (See, e.g., Muyldermans, S., 2001, J. Biotechnol. 74:277-302) The reduced-size antigen binding fragments are well expressed in bacteria, bind to antigen with high affinity, and are very stable. Phage display libraries of single domain antibodies (i.e., having a single variable domain that can be a light chain or a heavy chain variable domain) can be produced and screened in the same manner as scFv and Fab libraries. Scaffolds for such single domain antibodies can be modified mouse or human variable domains. It is noted that single antibody domains can bind antigen in a variety of antigen binding modes. That is, the primary antibody-antigen interactions are not limited to amino acid residues corresponding to CDRs of V_{H}-V_{L} containing antibodies, and consideration can be given to binding interactions outside of CDR residues when optimizing the binding characteristics of such antibodies.

The antibodies of the present invention bind to VEGF receptors preferably with an affinity comparable to or greater than that of the natural ligand. Affinity, represented by the equilibrium constant for the association of an antigen with an immunoglobulin molecule (K), measures the binding strength between and antigenic determinant and an antigen binding site, irrespective of the number of binding sites. K_{d}, the dissociation constant, is the reciprocal of K. An antigenic determinant, also known as an epitope, is the site on an antigen at which a given antibody binds. Typical values of K_{d} are 10⁻⁵ M to 10⁻¹¹ M. Any K_{d} greater than 10⁻⁴ M is considered to be non-specific binding.

Avidity is a measure of the strength of binding between an immunoglobulin and its antigen. Unlike affinity, which measures the strength of binding at each binding site, avidity is determined by both the affinity and the number of antigen specific binding sites (valency) of an immunoglobulin molecule.

The antibodies of the invention may comprise only immunoglobulin variable domains, optionally linked by amino acid sequences of synthetic origin. For example, a typical diabody has two Fv domains and comprises two chains—the first chain incorporating the heavy chain variable domain of a first antibody linked to the light chain variable domain of a second antibody, and the second chain comprising the light chain variable domain of the first antibody linked to the heavy chain variable domain of the second antibody. The domains are typically connected by a flexible polypeptide linker of about 5 to 10 amino acid residues, such as, for example, the 5 amino acid sequence Gly-Gly-Gly-Gly-Ser or the 10 amino acid sequence (Gly-Gly-Gly-Gly-Ser)₂. Pairing of first and second chains is favored over pairing of like chains, and a substantially homogeneous population of diabodies is achieved.

In certain embodiments, antibodies of the invention further comprise immunoglobulin constant regions of one or more immunoglobulin classes. Immunoglobulin classes include IgG, IgM, IgA, IgD, and IgE isotypes and, in the case of IgG and IgA, their subtypes. In a preferred embodiment, an antibody of the invention has a constant domain structure of an IgG type antibody, but has four antigen binding sites. This is accomplished by substituting a complete antigen binding sites (e.g., a single chain Fv) for each of the immunoglobulin variable domains. The four antigen-binding sites preferably comprise two binding sites for each of two different binding specificities.

An antigen binding site for inclusion in an antibody having desired binding characteristics is obtained by a variety of methods. The amino acid sequences of the V_{L} and/or V_{H} portions of a selected binding domain correspond to a naturally-occurring antibody or are chosen or modified to obtained desired immunogenic or binding characteristics. For example, V_{L} and V_{H} domains can be obtained directly from a monoclonal antibody which has the desired binding characteristics. Anti-VEGFR-2 monoclonal antibodies include DC101 (rat anti-mouse VEGFR-2; deposited as ATCC HB 11534), M25.18 A1 (mouse anti-mouse VEGFR-2; deposited as ATCC HB 12152), and M73.24 (mouse anti-mouse VEGFR-2; deposited as ATCC HB 12153). Anti-VEGFR-1 monoclonal antibodies include KM1730 (deposited as FERM BP-5697), KM1731 (deposited as FERM BP-5718), KM1732 (deposited as FERM BP-5698), KM1748 (deposited as FERM BP-5699), and KM1750 (deposited as FERM BP-5700), disclosed in WO 98/22616, WO 99/59636, Australian accepted application no. AU 1998 50666 B2, and Canadian application no. CA 2328893.

Alternatively, V_{L} and V_{H} domains can be from libraries of V gene sequences from a mammal of choice. Elements of such libraries express random combinations of V_{L} and/or V_{H} domains and are screened with any desired antigen to identify those elements which have desired binding characteristics. Particularly preferred is a human V gene library. Methods for such screening are known in the art. V_{L} and V_{H} domains from a selected non-human source may be incorporated into chimeric antibodies. For example, for administration to a human, it may be desired to use a bispecific antibody with functional constant domains wherein the V_{L} and V_{H} domains have been selected from a non-human source. To maximize constant domain associated function or to reduce immunogenicity of the antibody, human constant regions are preferred.

Alternatively, a bispecific antibody can be made that is “humanized.” Humanized variable domains are constructed in which amino acid sequences which comprise one or more complementarity determining regions (CDRs) of non-human origin are grafted to human framework regions (FRs). For examples, see: Jones, P. T. et al., (1996) Nature 321, 522-525; Riechman, L. et al., (1988) Nature 332, 323-327; U.S. Pat. No. 5,530,101 to Queen et al. A humanized construct is particularly valuable for elimination of adverse immunogenic characteristics, for example, where an antigen binding domain from a non-human source is desired to be used for treatment in a human. Variable domains have a high degree of structural homology, allowing easy identification of amino acid residues within variable domains which corresponding to CDRs and FRs. See, e.g., Kabat, E. A., et al. (1991) Sequences of Proteins of Immunological Interest. 5th ed. National Center for Biotechnology Information, National Institutes of Health, Bethesda, Md. Thus, amino acids which participate in antigen binding are easily identified. In addition, methods have been developed to preserve or to enhance affinity for antigen of humanized binding domains comprising grafted CDRs. One way is to include in the recipient variable domain the foreign framework residues which influence the conformation of the CDR regions. A second way is to graft the foreign CDRs onto human variable domains with the closest homology to the foreign variable region. Queen, C. et al., (1989) Proc. Natl. Acad. Sci. USA 86,10029-10033. CDRs are most easily grafted onto different FRs by first amplifying individual FR sequences using overlapping primers which include desired CDR sequences, and joining the resulting gene segments in subsequent amplification reactions. Grafting of a CDR onto a different variable domain can further involve the substitution of amino acid residues which are adjacent to the CDR in the amino acid sequence or packed against the CDR in the folded variable domain structure which affect the conformation of the CDR. Humanized domains of the invention therefore include human antibodies which comprise one or more non-human CDRs as well as such domains in which additional substitutions or replacements have been made to preserve or enhance binding characteristics.

Antibodies of the invention also include antibodies which have been made less immunogenic by replacing surface-exposed residues to make the antibody appear as self to the immune system (Padlan, E. A. (1991) Mol. Immunol. 28, 489-498). Antibodies have been modified by this process with no loss of affinity Roguska et al. (1994) Proc. Natl. Acad. Sci USA 91, 969-973). Because the internal packing of amino acid residues in the vicinity of the antigen binding site remains unchanged, affinity is preserved. Substitution of surface-exposed residues according to the invention for the purpose of reduced immunogenicity does not mean substitution of CDR residues or adjacent residues which influence binding characteristics.

The invention contemplates binding domains which are essentially human. Human binding domains can be obtained from phage display libraries wherein human heavy or light chain variable domains or combinations thereof are displayed on the surface of filamentous phage (See, e.g., McCafferty et al. (1990) Nature 348, 552-554; Aujame et al. (1997)Human Antibodies 8, 155-168). Combinations of variable domains are typically displayed on filamentous phage in the form of Fabs or scFvs. The library is screened for phage bearing combinations of variable domains having desired antigen binding characteristics. Preferred variable domains and variable domain combinations display high affinity for a selected antigen and little cross-reactivity to other related antigens. By screening very large repertoires of antibody fragments, (see e.g., Griffiths et al. (1994) EMBO J. 13, 3245-3260) a good diversity of high affinity Mabs are isolated, with many expected to have sub-nanomolar affinities for the desired antigen.

Alternatively, human binding domains can be obtained from transgenic animals into which unrearranged human Ig gene segments have been introduced and in which the endogenous mouse Ig genes have been inactivated (reviewed in Brüggemann and Taussig (1997) Curr. Opin. Biotechnol. 8, 455-458). Preferred transgenic animals contain very large contiguous Ig gene fragments that are over 1 Mb in size (Mendez et al. (1997) Nature Genet. 15, 146-156) but human Mabs of moderate affinity can be raised from transgenic animals containing smaller gene loci (See, e.g., Wagner et al. (1994) Eur. J. Immunol. 42,2672-2681; Green et al. (1994) Nature Genet. 7, 13-21).

In a physiological immune response, mutation and selection of expressed antibody genes leads to the production of antibodies having high affinity for their target antigen. The V_{L} and V_{H} domains incorporated into antibodies of the invention can similarly be subject to in vitro mutation and screening procedures to obtain high affinity variants.

Binding domains of the invention include those for which binding characteristics have been improved by direct mutation or by methods of affinity maturation. Affinity and specificity may be modified or improved by mutating CDRs and screening for antigen binding sites having the desired characteristics (See, e.g., Yang et al. (1995) J. Mol. Bio. 254, 392-403). CDRs are mutated in a variety of ways. One way is to randomize individual residues or combinations of residues so that in a population of otherwise identical antigen binding sites, all twenty amino acids, or a subset thereof, are found at particular positions. Alternatively, mutations are induced over a range of CDR residues by error prone PCR methods (See, e.g., Hawkins et al. (1992) J. Mol. Bio. 226, 889-896). Phage display vectors containing heavy and light chain variable region genes are propagated in mutator strains of E. coli (See, e.g., Low et al. (1996) J. Mol. Bio. 250, 359-368). These methods of mutagenesis are illustrative of the many methods known to one of skill in the art.

Each variable domain of the antibodies of the present invention may be a complete immunoglobulin heavy or light chain variable domain, or it may be a functional equivalent or a mutant or derivative of a naturally occurring domain, or a synthetic domain constructed, for example, in vitro using a technique such as one described in WO 93/11236 (Medical Research Council et al./Griffiths et al.). For instance, it is possible to join together domains corresponding to portions of antibody variable domains (i.e., variable domains which are missing at least one amino acid). The important characterizing feature is the ability of each variable domain to associate with a complementary variable domain to form an antigen binding site.

In another aspect of the invention, the antibodies can be chemically or biosynthetically linked to anti-tumor agents or detectable signal-producing agents. Anti-tumor agents linked to an antibody include any agents which destroy or damage a tumor to which the antibody has bound or in the environment of the cell to which the antibody has bound. For example, an anti-tumor agent is a toxic agent such as a chemotherapeutic agent or a radioisotope. Suitable chemotherapeutic agents are known to those skilled in the art and include anthracyclines (e.g. daunomycin and doxorubicin), methotrexate, vindesine, neocarzinostatin, cis-platinum, chlorambucil, cytosine arabinoside, 5-fluorouridine, melphalan, ricin and calicheamicin. The chemotherapeutic agents are conjugated to the antibody using conventional methods (See, e.g., Hermentin and Seiler (1988) Behring Inst. Mitt. 82, 197-215).

Detectable signal-producing agents are useful in vivo and in vitro for diagnostic purposes. The signal producing agent produces a measurable signal which is detectible by external means, usually the measurement of electromagnetic radiation. For the most part, the signal producing agent is an enzyme or chromophore, or emits light by fluorescence, phosphorescence or chemiluminescence. Chromophores include dyes which absorb light in the ultraviolet or visible, region, and can be substrates or degradation products of enzyme catalyzed reactions.

The invention further contemplates antibodies to which target or reporter moieties are linked. Target moieties are first members of binding pairs. Anti-tumor agents, for example, are conjugated to second members of such pairs and are thereby directed to the site where the antibody is bound. A common example of such a binding pair is avidin and biotin. In a preferred embodiment, biotin is conjugated to an antibody of the invention, and thereby provides a target for an anti-tumor agent or other moiety which is conjugated to avidin or streptavidin. Alternatively, biotin or another such moiety is linked to an antibody of the invention and used as a reporter, for example in a diagnostic system where a detectable signal-producing agent is conjugated to avidin or streptavidin.

Suitable radioisotopes for use as anti-tumor agents are also known to those skilled in the art. For example, ¹³¹I or ²¹¹ At is used. These isotopes are attached to the antibody using conventional techniques (See, e.g., Pedley et al. (1993) Br. J. Cancer 68, 69-73). Alternatively, the anti-tumor agent which is attached to the antibody is an enzyme which activates a prodrug. In this way, a prodrug is administered which remains in its inactive form until it reaches the tumor site where it is converted to its cytotoxin form once the antibody complex is administered. In practice, the antibody-enzyme conjugate is administered to the patient and allowed to localize in the region of the tissue to be treated. The prodrug is then administered to the patient so that conversion to the cytotoxic drug occurs in the region of the tissue to be treated. Alternatively, the anti-tumor agent conjugated to the antibody is a cytokine such as interleukin-2 (IL,2), interleukin-4 (IL-4) or tumor necrosis factor alpha (TNF-α). The antibody targets the cytokine to the tumor so that the cytokine mediates damage to or destruction of the tumor without affecting other tissues. The cytokine is fused to the antibody at the DNA level using conventional recombinant DNA techniques.

The proteins of the invention can be fused to additional amino acid residues such as a peptide tag to facilitate isolation or purification, or a signal sequence to promote secretion or membrane transport in any particular host in which the protein is expressed.

Vectors for construction and expression of antibodies of the invention in bacteria are available which contain secretion signal sequences and convenient restriction cloning sites. V_{L} and V_{H} gene combinations encoding binding sites specific for a particular antigen are isolated from cDNA of B cell hybridomas. Alternatively, random combinations of V_{L} and V_{H} genes are obtained from genomic DNA and the products then screened for binding to an antigen of interest. Typically, the polymerase chain reaction (PCR) is employed for cloning, using primers which are compatible with restriction sites in the cloning vector. See, e.g., Dreher, M. L. et al. (1991) J. Immunol. Methods 139:197-205; Ward, E. S. (1993) Adv. Pharmacol. 24:1-20; Chowdhury, P. S. and Pastan, I. (1999) Nat. Biotechnol. 17:568-572.

To express antibodies with selected or random combinations of V_{L} and V_{H} domains, V genes encoding those domains are assembled into a bacterial expression vector. For example, a vector can be used which has sequences encoding a bacterial secretion signal sequence and a peptide linker and which has convenient restriction sites for insertion of V_{L} and V_{H} genes. Alternatively, it might be desired to first assemble all necessary coding sequences (e.g., secretion signal, V_{L}, V_{H} and linker peptide) into a single sequence, for example by PCR amplification using overlapping primers, followed by ligation into a plasmid or other vector. Where it is desired to provide a specific combination of V_{L} and V_{H} domains, PCR primers specific to the sequences encoding those domains are used. Where it is desired to create a diverse combinations of a large number of V_{L} and V_{H} domain, mixtures of primers are used which amplify multiple sequences.

Preferred diabodies of the invention are made by expressing 1) a first polypeptide comprising a heavy chain variable domain corresponding to a first specificity connected to a light chain variable domain of a second specificity; and 2) a second polypeptide comprising a light chain variable domain corresponding to the first specificity connected to the heavy chain variable domain of to the second specificity. Diabodies are commonly produced in E. coli using DNA constructs which comprise bacterial secretion signal sequences at the start of each polypeptide chain.

For certain binding proteins of the invention, expression in other host cells may be desired. For example, binding proteins comprising constant domains are often more efficiently expressed in eukaryotic cells, including yeast, insect, vertebrate and mammalian cells. It will be necessary to use such cells where it is desired that the expressed product be glycosylated. The DNA fragments coding for the first and second polypeptides can be cloned, e.g., into HCMV vectors designed to express human light chains of human heavy chains in mammalian cells. (See, eg., Bendig, et al., U.S. Pat. No. 5,840,299; Maeda, et al. (1991) Hum. Antibod. Hybridomas 2, 124-134). Such vectors contain the human cytomegalovirus (HCMV) promoter and enhancer for high level transcription of the light chain and heavy chain constructs. In a preferred embodiment, the light chain expression vector is pKN100 (gift of Dr. S. Tarran Jones, MRC Collaborative Center, London, England), which encodes a human kappa light chain, and the heavy chain expression vector is pG1D105 (gift of Dr. S. Tarran Jones), which encodes a human gamma-1 heavy chain. Both vectors contain HCMV promoters and enhancers, replication origins and selectable markers functional in mammalian cells and E. coli.

A selectable marker is a gene which encodes a protein necessary for the survival or growth of transformed host cells grown in a selective culture medium. Typical selectable markers encode proteins that (a) confer resistance to antibiotics or other toxins, e.g. ampicillin, neomycin, methotrexate, or tetracycline, (b) complement auxotrophic deficiencies, or (c) supply critical nutrients not available from complex media, e.g. the gene encoding D-alanine racemase for Bacilli. A particularly useful selectable marker confers resistance to methotrexate. For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity, prepared and propagated as described by Urlaub and Chasin (1980) Proc. Natl. Acad. Sci. USA 77, 4216. The transformed cells are then exposed to increased levels of methotrexate. This leads to the synthesis of multiple copies of the DHFR gene, and, concomitantly, multiple copies of other DNA comprising the expression vectors, such as the DNA encoding the antibody or antibody fragment. In another example, mutant myeloma cells that are deficient for thymidine kinase (TK) are unable to use exogenously supplied thymidine when aminopterin is used to block DNA synthesis. Useful vectors for transfection carry an intact TK gene which allows growth in media supplemented with thymidine.

Where it is desired to express a gene construct in yeast, a suitable selection gene for use in yeast is the trp1 gene present in the yeast plasmid YRp7. Stinchcomb et al., 1979 Nature, 282, 39; Kingsman et al., 1979, Gene 7, 141. The trp1 gene provides a selection marker for a mutant strain of yeast lacking the ability to grow in tryptophan, for example, ATCC No.44076 or PEP4-1. Jones (1977) Genetics 85,12. The presence of the tip I lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

Preferred host cells for transformation of vectors and expression of antibodies of the present invention are bacterial cells, yeast cells and mammalian cells, e.g., COS-7 cells, chinese hamster ovary (CHO) cells, and cell lines of lymphoid origin such as lymphoma, myeloma, or hybridoma cells. The transformed host cells are cultured by methods known in the art in a liquid medium containing assimilable sources of carbon, e.g. carbohydrates such as glucose or lactose, nitrogen, e.g. amino acids, peptides, proteins or their degradation products such as peptones, ammonium salts or the like, and inorganic salts, e.g. sulfates, phosphates and/or carbonates of sodium, potassium, magnesium and calcium. The medium furthermore contains, for example, growth-promoting substances, such as trace elements, for example iron, zinc, manganese and the like.

Antibodies of the instant invention have dual specificity and capable of binding to two different antigens simultaneously. The different antigens can be located on different cells or on the same cell. Cross linking of antigen can be shown in vitro, for example by providing a solid surface to which a first antigen has been bound, adding a bispecific antibodies specific for the first antigen and a second antigen for which the binding protein is also specific and detecting the presence of bound second antigen.

Antibodies of the invention can of block the interaction between two receptors and their respective ligands. For example, a diabody specific for KDR and Flt- 1 inhibits VEGF induced cell migration as well as PlGF induced cell migration. In this case, combination of two receptor binding specificities, either as a mixture of single chains antibodies (scFvs) or in a bispecific diabody, is more efficacious in inhibiting cell migration that the individual parent antibodies.

Compared to antibodies that are monospecific, bispecific antibodies can be more potent inhibitors of cellular function. For example, VEGF-stimulated cellular functions such as, for example, proliferation of endothelial cells and VEGF- and PlGF-induced migration of human leukemia cells can be more efficiently inhibited by bispecific antibodies, even where affinity for one or both of the two target antigens is reduced. In one embodiment of the invention, a diabody was made that was specific for KDR and Flt-1. scFv corresponding to either of the target antigens was unable to completely inhibit VEGF- or PlGF-induced cell migration, even at the highest scFv concentrations tested. In contrast, a diabody specific for both of the target antigens completely abolished cell migration, even though the affinity of the diabody for Flt-1 was reduced compared to the corresponding scFv.

The antibodies of the present invention are useful for treating diseases in humans and other mammals. The antibodies are used for the same purposes and in the same manner as heretofore known for natural and engineered antibodies. The present antibodies thus can be used in vivo and in vitro for investigative, diagnostic or treatment methods which are well known in the art.

The present antibodies can be administered for therapeutic treatments to a patient suffering from a tumor in an amount sufficient to prevent or reduce the progression of the tumor, e.g, the growth, invasiveness, metastases and/or recurrence of the tumor. An amount adequate to accomplish this is defined as a therapeutically effective dose. Amounts effective for this use will depend upon the severity of the disease and the general state of the patient's own immune system. Dosing schedules will also vary with the disease state and status of the patient, and will typically range from a single bolus dosage or continuous infusion to multiple administrations per day (e.g., every 4-6 hours), or as indicated by the treating physician and the patient's condition. Antibodies of the invention can be administered in a single dosages as high as 40 mg/kg body-weight or higher. More preferably, the antibodies are administered in dosages that range from 0.2 mg/kg to 20 mg/kg body-weight. It should be noted, however, that the present invention is not limited to any particular dose.

The present invention can be used to treat any suitable tumor, including, for example, tumors of the breast, heart, lung, small intestine, colon, spleen, kidney, bladder, head and neck, ovary, prostate, brain, pancreas, skin, bone, bone marrow, blood, thymus, uterus, testicles, cervix or liver. Tumors of the present invention preferably have aberrant expression or signaling of VEGFR. Enhanced signaling by VEGFR has been observed in many different human cancers. High levels of VEGFR-2 are expressed by endothelial cells that infiltrate gliomas (Plate, K. et al., (1992) Nature 359:845-848). VEGFR-2 levels are specifically upregulated by VEGF produced by human glioblastomas (Plate, K. et al. (1993) Cancer Res. 53:5822-5827). The finding of high levels of VEGFR-2 expression in glioblastoma associated endothelial cells (GAEC) indicates that receptor activity is probably induced during tumor formation since VEGFR-2 transcripts are barely detectable in normal brain endothelial cells. This upregulation is confined to the vascular endothelial cells in close proximity to the tumor.

The antibodies of the invention are also to be used in combined treatment methods. The bispecific antibodies can be administered with an anti-neoplastic agent such as a chemotherapeutic agent or a radioisotope. Suitable chemotherapeutic agents are known to those skilled in the art and include anthracyclines (e.g. daunomycin and doxorubicin), paclitaxel, irinotecan (CPT-11), topotecan, methotrexate, vindesine, neocarzinostatin, cisplatin, chlorambucil, cytosine arabinoside, 5-fluorouridine, melphalan, ricin, calicheamicin, and combinations thereof. A bispecific antibody and an anti-neoplastic agent are admininstered to a patient in amounts effective to inhibit angiogenesis and reduce tumor growth. The antibodies are also to be administered in combination with other treatment regimes. For example, bispecific antigen binding proteins of the invention can be administered with radiation, either external (external beam radiation therapy) or internal (brachytherapy).

It is understood that antibodies of the invention, where used in the human body for the purpose of diagnosis or treatment, will be administered in the form of a composition additionally comprising a pharmaceutically-acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of this invention may be in a variety of forms. These include, for example, solid, semi-solid and liquid dosage forms, such as tablets, pills, powders, liquid solutions, dispersions or suspensions, liposomes, suppositories, injectable and infusible solutions. The preferred form depends on the intended mode of administration and therapeutic application. The preferred compositions are in the form of injectable or infusible solutions.

Therapeutic compositions of this invention are similar to those generally used for passive immunization of humans with antibodies as are known to those of skill in the art, and include but are not limited to intraveneous, intraperitoneal, subsutaneous, and intramuscular administration. Further, it is understood that combination treatments may involve administration of antibodies and, e.g., chemotherapeutic agents, by different methods.

It is to be understood and expected that variations in the principles of invention herein disclosed may be made by one skilled in the art and it is intended that such modifications are to be included within the scope of the present invention.

The examples which follow further illustrate the invention, but should not be construed to limit the scope of the invention in any way. Detailed descriptions of conventional methods, such as those employed in the construction of vectors and plasmids, the insertion of genes encoding polypeptides into such vectors and plasmids, the introduction of plasmids into host cells, and the expression and determination thereof of genes and gene products can be obtained from numerous publication, including Sambrook, J. et al., (1989) Molecular Cloning: A Laboratory Manual, 2^{nd} ed., Cold Spring Harbor Laboratory Press. All references mentioned herein are incorporated in their entirety.

### EXAMPLE 1

### Materials and Methods

Cell lines.

A hybridoma cell line (ATC No. PTA-334) producing the anti-Flt-1 antibody, Mab6.12 (IgGl, κ), was established at ImClone Systems Incorporated (New York, N.Y.) from a mouse immunized with a recombinant form of the receptor. Primary-cultured human umbilical vein endothelial cells (HUVEC) were obtained from Dr. S. Rafii at Cornell Medical Center, New York, and maintained in EBM-2 medium (Clonetics, Walkersville, Md.) at 37° C., 5% CO₂. The leukemia cell lines, HL60 and HEL, were maintained in RPMI containing 10% of fetal calf serum and grown at 37° C. with 5% CO₂.

### Proteins and Antibodies.

The soluble fusion protein KDR-alkaline phosphatase (AP) was expressed in stably transfected NIH 3T3 and purified from cell culture supernatant by affinity chromatography using immobilized monoclonal antibody to AP as described by Lu, D., et al., 2000, J. Biol. Chem., 275:14321-14330. VEGF165 protein was expressed in baculovirus and purified following the procedures described. Id. PlGF and Flt-1-Fc fusion proteins were purchased from R&D Systems (Minneapolis, Minn.).

Preparation of scFv Specific for Flt-1.

The V_{H} and V_{L} genes of Mab 6.12 were cloned by RT-PCR from mRNA isolated from the hybridoma cells, following the procedures of Bendig et al. (1996) In: Antibody Engineering: A Practical Approach, McCafferty, J., Hoogenboom, H. R., Chiswell, D. J., eds., Oxford University Press, Incorporated; p147-168. Eleven 5′ primers, specifically designed to hybridize to the 5′ ends of mouse antibody light chain leader sequences, and one 3′ primer that hybridizes to the 5′ end of mouse κ light chain constant region, were used to clone the V_{L} gene. Twelve 5′ primers, specifically designed to hybridize to the 5′ ends of mouse antibody heavy chain leader sequences, and one 3′ primer that hybridizes to the 5′ end of mouse IgGl heavy chain constant region were used to clone the V_{H} gene. In total, twenty-three PCR reactions, eleven for the V_{L} gene and twelve for the V_{H} gene, were carried out. All PCR-generated fragments with sizes between 400 to 500 base pairs were cloned into the pCR® 2.1 vector as described in the manufacturer's instruction (TA Cloning® Kit, Invitrogen, Carlsbad, Calif.), followed by transformation of E.coli strain, XL-1.

PCR fragments encoding the V_{L} and the V_{H} genes of MAB 6.12 were used to assemble scFv 6.12, using overlapping PCR. In this scfv, the C-terminal of Mab 6.12 V_{H} is linked to the N-terminal of Mab 6.12 V_{L} via a 15 amino acid linker, (Glycine-Glycine-Glycine-Glycine-Serine)₃, or (GGGGS)₃ (FIG. 1A). The scFv 6.12-encoding gene was then cloned into vector pCANTAB 5E (Amersham Pharmacia Biotech, Piscataway, N.J.) for the expression of the soluble scFv protein. The amino acid and nucleotide sequences for the Mab 6.12 V_{H} domain are given by SEQ ID NOS:41 and 49, respectively. Similarly, the amino acid and nucleotide sequences for the Mab 6.12 V_{L} domain are presented by SEQ ID NOS:42 and 50. Amino acid sequences for CDRH1, CDRH2, CDRH3, CDRL1, CDRL2, and CDRL2 are presented by SEQ ID NOS:35, 36,37, 38, 39, and 40, respectively. The corresponding nucleotide sequences are presented by SEQ ID NOS:43 to 48.

Preparation and Biopanning of scFv Specific for KDR.

A single chain antibody directed against KDR, scFv p1C11, was isolated from a phage display library constructed from the splenocytes of a mouse immunized with KDR (Zhu, Z. et al., 1998, Cancer Res. 58:3209-3214). Female BALB/C mice were given two intraperitoneal (i.p.) injections of 10 μg KDR-AP in 200 μl of RIBI Adjuvant System followed by one i.p. injection without RIBI adjuvant over a period of two months. The mice were also given a subcutaneous (s.c.) injection of 10 μg KDR-AP in 200 μl of RIBI at the time of the first immunization. The mice were boosted i.p. with 20 μg of KDR-AP three days before euthanasia. mRNA was purified from total RNA extracted from splenocytes. Following reverse transcription, cDNAs corresponding to expressed V_{L} and V_{H} genes were separately amplified. The amplified products were inserted into a vector designed to accept each gene separately or linked to nucleotides encoding a secretion signal sequence and polypeptide linker (e.g., by PCR amplification) and the fused product inserted into a desired vector. See, e.g., Zhu et al., 1998.

To display the scFv on filamentous phage, antibody V_{H} and V_{L} domains were joined by a 15 amino acid linker (GGGGS)₃. The C terminus of this construct was joined to the N terminus of phage protein III with a 15 amino-acid E tag, ending with an amber codon (TAG). The amber codon positioned between the E tag and protein III allows production of scFv in soluble form when transformed into a nonsupressor host (e.g., HB2151 cells), and phage display via protein m when transformed into a suppressor host (e.g., TG1 cells).

The scFv-gene m constructs were ligated into the pCANTAB 5E vector. Transformed TG1 cells were plated onto 2YTAG plates (17 g/l tryptone, 10 g/l yeast extract, 5 g/l NaCl, 20 g/l glucose, 100 μg/ml ampicillin, 15 g/l Bacto-agar) and incubated. The colonies were scraped into 10 ml of 2YT medium (17 g/l tryptone, 10 g/l yeast extract, 5 g/l NaCl), mixed with 5 ml 50% glycerol and stored at −70° C. as the library stock.

The library stock was grown to log phase, rescued with M13K07 helper phage and amplified overnight in 2YTAK medium (2YT containing 100 /μg/ml of ampicillin and 50 μg/ml of kanamycin) at 30° C. The phage preparation was precipitated in 4% PEG/0.5 M NaCl, resuspended in 3% fat-free milk/PBS containing 500 μg/ml of alkaline phosphatase (AP) and incubated at 37° C. for 1 h to block phage-scFv having specificity for AP scFv and to block other nonspecific binding.

KDR-AP (10 μg/ml) coated Maxisorp Star tubes (Nunc, Denmark) were first blocked with 3% milk/PBS at 37° C. for 1 h, and then incubated with the phage preparation at room temperature for 1 h. The tubes were washed 10 times with PBST (PBS containing 0.1% Tween 20), followed by 10 times with PBS. The bound phage were eluted at room temperature for 10 min. with 1 ml of a freshly prepared solution of 100 mM triethylamine. The eluted phage were incubated with 10 ml of mid-log phase TG1 cells at 37° C. for 30 min. stationary and 30 min. shaking. The infected TG1 cells were then plated onto 2YTAG plates and incubated overnight at 30° C. as provided above for making of the phage stock.

Successive rounds of the screening procedure were employed to further enrich for displayed scFv having the desired binding specificity. After two or three rounds of panning, individual bacterial colonies were screened individually to identify clones having desired KDR binding characteristics. Identified clones were further tested for blocking of VEGF binding. DNA fingerprinting of clones was used to differentiate unique clones. Representative clones of each digestion pattern were picked and subject to DNA sequencing.

### Human Antibodies Specific for KDR.

A large human Fab phage display library containing 3.7×10¹⁰ clones (DeHaard et al., J. Biol. Chem. 274:18218-30 (1999)) was used for the selection. The library consists of combinations of PCR-amplified antibody variable light chain genes fused to human constant chain genes (κ and λ) and variable heavy chain genes fused to DNA encoding the human IgG1 heavy chain C_{H}1 domain. Both heavy and light chain constructs are preceded by a signal sequence—pelB for the light chain and gene III signal sequence for the heavy chain. Heavy chain constructs further encode a portion of the gene III protein for phage display, a hexahistidine tag, and an 11 amino-acid-long c-myc tag, followed by an amber codon (TAG). The hexahistidine and c-myc tags can be used for purification or detection. The amber codon allows for phage display using suppressor hosts (such as TG1 cells) or production of Fab fragments in soluble form when transformed into a nonsupressor host (such as HB2151 cells).

The library stock was grown to log phase, rescued with M13-KO7 helper phage and amplified overnight in 2YTAK medium (2YT containing 100 μg/ml of ampicillin and 50 μg/ml of kanamycin) at 30° C. The phage preparation was precipitated in 4% PEG/0.5 M NaCl, resuspended in 3% fat-free milk/PBS containing 500 μg/ml of AP protein and incubated at 37° C. for 1 h to capture phage displaying anti-AP Fab fragments and to block other nonspecific binding.

KDR-AP (10 μg/ml in PBS) coated Maxisorp Star tubes (Nunc, Rosklide, Denmark) were first blocked with 3% milk/PBS at 37° C. for 1 h, and then incubated with the phage preparation at RT for 1 h. The tubes were washed 10 times with PBST (PBS containing 0.1% Tween-20) followed by 10 times with PBS. Bound phage were eluted at RT for 10 min with 1 ml of a freshly prepared solution of 100 mM triethylamine (Sigma, St. Louis, Mo.). The eluted phage were incubated with 10 ml of mid-log phase TG1 cells at 37° C. for 30 min stationary and 30 min shaking. The infected TG1 cells were pelleted and plated onto several large 2YTAG plates and incubated overnight at 30° C. All the colonies grown on the plates were scraped into 3 to 5 ml of 2YTA medium, mixed with glycerol (10% final concentration), aliquoted and stored at −70° C. For the next round selection, 100 μl of the phage stock was added to 25 ml of 2YTAG medium and grown to mid-log phase. The culture was rescued with M13K07 helper phage, amplified, precipitated, and used for selection followed the procedure described above, with reduced concentrations of KDR-AP immobilized on the immunotube and increased number of washes after the binding process.

A total of three rounds of selection were performed on immobilized KDR, with varying protein concentrations and number of washings after the initial binding process. After each round selection, 93 clones were randomly picked and tested by phage ELISA for binding to KDR. Seventy out of the 93 clones (75%) picked after the second selection, and greater than 90% of the recovered clones after the third selection were positive in KDR binding, suggesting a high efficiency of the selection process. DNA segments encoding the Fab from all the 70 binders identified in the second selection were amplified, digested with BstN I, and compared for fingerprint patterns. A total of 42 different patterns were observed, indicating an excellent diversity of the isolated anti-KDR Fab. Cross-reactivity examination demonstrated that 19 out of the 42 antibodies were specific KDR-binders, whereas the rest 23 antibodies bound to both KDR and its murine homologue, Flk-1. Further selection was achieved with a competitive VEGF-binding assay in which the binding of soluble KDR to immobilized VEGF in the presence or absence of the anti-KDR Fab fragments was determined. The assay identified four Fab clones that were capable of blocking the binding between VEGF and KDR. Three were KDR-specific binders and one cross-reacted with Flk-1. DNA fingerprinting and sequencing analysis confirmed that all four KDR/VEGF blocking antibodies were different (FIG. 1A) with unique DNA and amino acid sequences.

The amino acid sequences for CDR1, CDR2 and CDR3 of V_{H} and V_{L} for the four clones are given in Table 1.

TABLE 1

CDR sequences of selected KDR-binding human Fabs

| | | | |
|---|---|---|---|
| Clone | CDR1 | CDR2 | CDR3 |

Light Chain

| | | | |
|---|---|---|---|
| D2C6 | RASQSVSSYLA | DSSNRAT | LQHNTFPPT |
| | (SEQ ID NO: 53) | (SEQ ID NO: 54) | (SEQ ID NO: 55) |
| D2H2 | RASQGISSRLA | AASSLQT | QQANRFPPT |
| | (SEQ ID NO: 56) | (SEQ ID NO: 57) | (SEQ ID NO: 58) |
| D1H4 | AGTTTDLTYYDLVS | DGNKRPS | NSYVSSRFYV |
| | (SEQ ID NO: 59) | (SEQ ID NO: 60) | (SEQ ID NO: 61) |
| D1F7 | SGSTSNIGTNTAN | NNNQRPS | AAWDDSLNGHWV |
| | (SEQ ID NO: 62) | (SEQ ID NO: 63) | (SEQ ID NO: 64) |

Heavy Chain

| | | | |
|---|---|---|---|
| D2C6 | GFTFSSYSMN | SISSSSSYIYYADSVKG | VTDAFDI |
| | (SEQ ID NO: 65) | (SEQ ID NO: 66) | (SEQ ID NO: 67) |
| D2H2 | GFTFSSYSMN | SISSSSSYIYYADSVKG | VTDAFDI |
| D1H4 | GFTFSSYSMN | SISSSSSYIYYADSVKG | VTDAFDI |
| D1F7 | GGTFSSYAIS | GGIIPIFGTANYAQKFQG | GYDYYDSSGVASPFDY |
| | (SEQ ID NO: 68) | (SEQ ID NO: 69) | (SEQ ID NO: 70) |

Complete sequences for the V_{H} and V_{L} chains are presented in the Sequence Listing as follows. D1F7: V_{H} nucleotide and amino acid sequences in SEQ ID NOS:71 and 72; V_{L} nucleotide and amino acid sequences in SEQ ID NOS:73 and 74. D2C6: V_{H} nucleotide and amino acid sequences in SEQ ID NOS:75 and 76; V_{L} nucleotide and amino acid sequences in SEQ ID NOS:77 and 78. D2H2: V_{H} nucleotide and amino acid sequences in SEQ ID NOS:82 and 83; V_{L} nucleotide and amino acid sequences in SEQ ID NOS:84 and 85. D1H4: V_{H} nucleotide and amino acid sequences in SEQ ID NOS:79 and 76; V_{L} nucleotide and amino acid sequences in SEQ ID NOS:80 and 81.

A second library, consisting of combinations of the single heavy chain of D2C6 with a diverse population of light chains derived from the original library, was created and screened. Ten additional Fabs were identified, designated SA1, SA3, SB10, SB5, SC7, SD2, SD5, SF2, SF7, and 1121. Complete V_{L} nucleotide and amino acid sequences are presented in the Sequence Listing as follows. SA1: V_{L} nucleotide and amino acid sequences in SEQ ID NOS:86 and 87. SA3: V_{L} nucleotide and amino acid sequences in SEQ ID NOS:88 and 89. SB10: V_{L} nucleotide and amino acid sequences in SEQ ID NOS:90 and 91. SB5: V_{L} nucleotide and amino acid sequences in SEQ ID NOS:92 and 93. SC7: V_{L} nucleotide and amino acid sequences in SEQ ID NOS:94 and 95. SD2: V_{L} nucleotide and amino acid sequences in SEQ ID NOS:96 and 97. SD5: V_{L} nucleotide and amino acid sequences in SEQ ID NOS:98 and 99. SF2: V_{L} nucleotide and amino acid sequences in SEQ ID NOS:100 and 101. SF7: V_{L} nucleotide and amino acid sequences in SEQ ID NOS:102 and 103. 1121: V_{L} nucleotide and amino acid sequences in SEQ ID NOS:104 and 105.

The V_{L} CDR sequences are presented in Table 2.

TABLE 2

Light chain CDR sequences of KDR-binding human Fabs

| | | | |
|---|---|---|---|
| Clone | CDR1 | CDR2 | CDR3 |

| | | | |
|---|---|---|---|
| SA1 | TGSHSNFGAGTDV | GDSNRPS | QSYDYGLRGWV |
| | (SEQ ID NO: 106) | (SEQ ID NO: 107) | (SEQ ID NO: 108) |
| SA3 | RASQNINNYLN | AASTLQS | QQYSRYPPT |
| | (SEQ ID NO: 109) | (SEQ ID NO: 110) | (SEQ ID NO: 111) |
| SB10 | TGSSTDVGNYNYIS | DVTSRPS | NSYSATDTLV |
| | (SEQ ID NO: 112) | (SEQ ID NO: 113) | (SEQ ID NO: 114) |
| SB5 | TGQSSNIGADYDVH | GHNNRPS | QSYDSSLSGLV |
| | (SEQ ID NO: 115) | (SEQ ID NO: 116) | (SEQ ID NO: 117) |
| SC7 | RASQDISSWLA | AASLLQS | QQADSFPPT |
| | (SEQ ID NO: 118) | (SEQ ID NO: 119) | (SEQ ID NO: 120) |
| SD2 | RASQSIKRWLA | AASTLQS | QQANSFPPT |
| | (SEQ ID NO: 121) | (SEQ ID NO: 122) | (SEQ ID NO: 123) |
| SD5 | SGSRSNIGAHYEVQ | GDTNRPS | QSYDTSLRGPV |
| | (SEQ ID NO: 124) | (SEQ ID NO: 125) | (SEQ ID NO: 126) |
| SF2 | TGSSSNIGTGYDVH | AYTNRPS | QSFDDSLNGLV |
| | (SEQ ID NO: 127) | (SEQ ID NO: 128) | (SEQ ID NO: 129) |
| SF7 | TGSHSNFGAGTDVH | GDTHRPS | QSYDYGLRGWV |
| | (SEQ ID NO: 130) | (SEQ ID NO: 131) | (SEQ ID NO: 132) |
| 1121 | RASQGIDNWLG | DASNLDT | QQAKAFPPT |
| | (SEQ ID NO: 133) | (SEQ ID NO: 134) | (SEQ ID NO: 135) |

### Construction of an Anti-KDR x Anti-Flt-1 Diabody.

To construct the diabody, variable domains of scFv p1C11 and scFv 6.12 were used for PCR-directed assembly to create the expression plasmid, pDAB-KF1 (FIG. 1A). First, the following gene fragments were generated by PCR from the V_{L} and V_{H} domains of p1C11 and MAB6.12: the V_{L} domain of p1C11 followed by a segment encoding a 5 amino-acid-linker, GGGGS; the V_{H} domain of MAB6.12 preceded by a segment encoding the GGGGS linker; the V_{L} domain of MAB6.12 preceded by a segment encoding the E. coli heat stable enterotoxin II (stII) signal sequence (Picken, R. N., et al., 1983, Infect. Immun. 42:269-275) and followed by a segment encoding the GGGGS linker; and the V_{H} domain of p1C11 preceded by a segment encoding the GGGGS linker. Cross-over scFv, pLH-1C11-6.12 and pLH-6.12-1C11, were constructed by annealing of PCR fragments p1C11 V_{L} and MAB6.12 V_{H}, and MAB6.12 V_{L} and p1C11 V_{H}, respectively, followed by PCR amplification to introduce appropriate restriction sites for subsequent cloning. The expression plasmid, pDAB-KF1, for co-secretion of the two cross-over scFv was constructed by ligation of the SfiI/NheI and the NheI/NotI fragments from pLH-1C11-6.12 and pLH-6.12-1C11, respectively, into vector pCANTAB 5E. All sequences encoding the cross-over scFv fragments were verified by DNA sequencing.

### Expression and Purification of the Diabody.

The diabody was prepared from E. coli strain HB2151 containing the expression plasmid grown at 30° C. in a shaker flask following the procedure previously described (Lu, D. et al., 1999, J. Immunol. Methods 230:159-171). A periplasmic extract of the cells was prepared by resuspending the cell pellet in 25 mM Tris (pH 7.5) containing 20% (w/v) sucrose, 200 mM NaCl, 1 mM EDTA and 0.1 mM PMSF, followed by incubation at 4° C. with gentle shaking for 1 h. After centrifugation at 15,000 rpm for 15 min, the soluble diabody was purified from the supernatant by anti-E tag affinity chromatography using the RPAS Purification Module (Amersham Pharmacia Biotech). To examine the purity of the diabody preparation, both the E. coli periplasmic extract and the purified diabody were electrophoresed in an 18% polyacrylamide gel (Novex, San Diego, Calif.) and visualized by staining with Colloidal Blue Stain kit (Novex).

### Dual Specificity of the Diabody to KDR and Flt-.

Two assays were carried out to determine the dual antigen binding capability of the diabody. First, a cross-linking assay was used to investigate whether the diabody is capable of binding both of its target antigens simultaneously. Briefly, the diabody or its parent scFv were first incubated in a 96-well Maxi-sorp microtiter plate (Nunc, Roskilde, Denmark) precoated with Flt-1-Fc fusion protein (1 μg/ml×100 ml per well overnight at 4° C.) at room temperature (RT) for 1 h. The plate was washed three times with PBS containing 0.1% Tween (PBST), followed by incubation with KDR-AP fusion protein at RT for additional 1 h. The plate-bound KDR-AP was then quantified by the addition of AP substrate, p-nitrophenyl phosphate (Sigma, St. Louis, Mo.), followed by reading of the absorbance at 405 nm (Lu, D. et al., 1999). In the second, direct binding assay, various amounts of diabody or scFv were added to KDR or Flt-1 coated 96-well plates and incubated at RT for 1 h, after which the plates were washed 3 times with PBST. The plates were then incubated at RT for 1 h with 100 μl of an anti-E tag antibody-HRP conjugate (Amersham Pharmacia Biotech). The plates were washed, peroxidase substrate added, and the absorbance at 450 nm read following the procedure described previously (Lu, D. et al., 1999).

VEGF/KDR, VEGF/Flt-1, aid PlGF/Flt-1 blocking assays.

The assays followed previously described protocols (Zhu, Z. et al., 1998; Lu, D. et al., 1999). Briefly, various amounts of the diabody or scFv were mixed with a fixed amount of KDR-AP (100 ng) or Flt-1-Fc fusion protein (50 ng) and incubated at RT for 1 h. The mixture were then transferred to 96-well microtiter plates precoated with VEGF₁₆₅ (200 ng/well) or PlGF (200 ng/well) and incubated at RT for an additional 2 h, after which the plates were washed 5 times with PBS. For the KDR-AP assay, the substrate for AP was added, followed by reading of the absorbance at 405 nm to quantify the plate-bound KDR-AP. For the Flt-1-Fc assay, the plate was incubated with a mouse anti-human Fc-HRP conjugate to quantify the plate-bound Flt-1-Fc. The IC₅₀, i.e., the antibody concentration required for 50% inhibition of KDR or Flt-1 binding to VEGF or PlGF, was then calculated.

### Analysis of Binding Kinetics.

The binding kinetics of the diabody and its parent scFv to KDR and Flt-1 were measured using a BIAcore biosensor (Pharmacia Biosensor). KDR-AP or Flt-1-Fc fusion protein was immobilized onto a sensor chip and soluble antibodies were injected at concentrations ranging from 1.5 nM to 100 nM. Sensorgrams were obtained at each concentration and were analyzed with, BIA Evaluation 2.0, a program to determine the rate constants kon and koff. The affinity constant, Kd, was calculated from the ratio of rate constants koff/kon.

### Anti-mitogenic Assay.

HUVEC (5×10³ cells/well) were plated onto 96-well tissue culture plates (Wallach, Inc., Gaithersburg, Md.) in 200 μl of EBM-2 medium without VEGF, basic fibroblast growth factor or epidermal growth factor (EGF) and incubated at 37° C. for 72 h. Various amounts of the antibodies were added to duplicate wells and pre-incubated at 37° C. for 1 h, after which VEGF165 was added to a final concentration of 16 ng/ml. After 18 h of incubation, 0.25 μCi of [³H]-TdR (Amersham) was added to each well and incubated for an additional 4 h. The cells were washed once with PBS, trypsinized and harvested onto a glass fiber filter (Printed Filtermat A, Wallach) with a cell harvester (Harvester 96, MACH III M, TOMTEC, Orange, Conn.). The membrane was washed three times with H₂O and air-dried. Scintillation fluid was added and DNA incorporated radioactivity was determined on a scintillation counter (Wallach, Model 1450 Microbeta Liquid Scintillation Counter).

### Leukemia Migration Assay.

HL60 and HEL cells were washed three times with serum-free plain RPMI 1640 medium and suspended in the medium at 1×10⁶/ml. Aliquots of 100 μl cell suspension were added to either 3-μm-pore transwell inserts (for HL60 cells), or 8-μm-pore transwell inserts (for HEL cells) (Costar®, Corning Incorporated, Corning, N.Y.) and incubated with the antibodies for 30 min at 37° C. The inserts were then placed into the wells of 24-well plates containing 0.5 ml of serum-free RPMI 1640 with or without VEGF165. The migration was carried out at 37° C., 5% CO₂ for 16-18 h for HL60 cells, or for 4 h for HEL cells. Migrated cells were collected from the lower compartments and counted with a Coulter counter (Model Z1, Coulter Electronics Ltd., Luton, England).

### EXAMPLE 2

### Anti-KDR x Anti-Flt-1 Diabody

### Diabody Structure.

An anti-KDR x anti-FIt-1 diabody made according to Example I was purified and analyzed by SDS-PAGE. The two component polypeptides were resolved under the electrophoretic conditions and gave rise to two major bands with mobility close to that anticipated (FIG. 1B); the lower band represents the first polypeptide (m.w., 25179.6 daltons), and the upper band correlates with the second polypeptide with E-tag (m.w., 26693.8 daltons) (FIG. 1A).

### Dual Specificity.

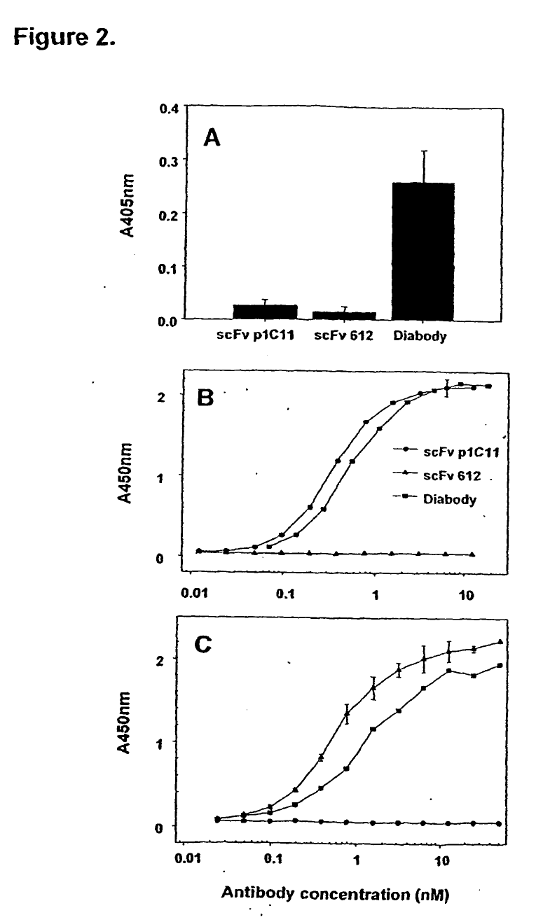

A cross-linking assay to investigate whether the anti-KDR x anti-Flt-1 diabody was capable of simultaneously binding to both of its target antigens. To test the capability of the Flt-1-bound diabody to capture soluble KDR, the diabody was first allowed to bind to immobilized Flt-1, followed by incubation with KDR-AP. As shown in FIG. 2A, the diabody, but not the parent monospecific scFv, efficiently cross-linked the soluble KDR to the immobilized Flt-1, as demonstrated by the plate-bound AP activity.

The antigen binding efficiency of the diabody was determined on immobilized KDR and Flt-1. The diabody bound as efficiently as the parent scFv p1C11 to KDR (FIG. 2B). Binding the diabody to Flt-1 was slightly reduced, compared to the parent scFv 6.12 (FIG. 2C). As expected, the KDR-specific scFv p1C11 did not bind to Flt-1 (FIG. 2B), and Flt-1-specific scFv 6.12 did not bind to KDR (FIG. 2C). Data shown in FIG. 2 represent the mean ±SD of triplicate samples.

The binding kinetics of the diabody to KDR and Fit-1 were determined by surface plasmon resonance using a BIAcore instrument (Table 3) and are consistent with the ELISA results of FIG. 2. The diabody binds to KDR with kinetics similar to its parent scFv p1C11 with a Kd of 1.4 nM. The binding affinity of the diabody to Flt-1 was moderately reduced compared to scFv 6.12, mainly due to a slower on-rate of the diabody (Table 3).

TABLE 3
Binding kinetics of the anti-KDR × anti-Flt-1 diabody as determined by BIAcore

| | KDR | | | Flt-1 | | |
|---|---|---|---|---|---|---|
| | kon | koff | | kon | koff | |
| Antibody | (10⁴ M⁻¹S⁻¹) | (10⁻⁴ S⁻¹) | Kd (nM) | (10⁴ M⁻¹S⁻) | (10⁻⁴ S⁻¹) | Kd (nM) |
| ScFv p1C11 | 7.42 ± 0.88 ^{a} | 1.21 ± 0.36 | 1.68 ± 0.66 | ND | ND | ND |
| ScFv 6.12 | ND | ND | ND | 24.1 ± 0.1 | 23.6 ± 4.8 | 9.8 ± 1.98 |
| Diabody | 6.24 ± 0.76 | 0.87 ± 0.14 | 1.40 ± 0.27 | 7.73 ± 1.15 | 23.4 ± 0.92 | 30.7 ± 5.7 |

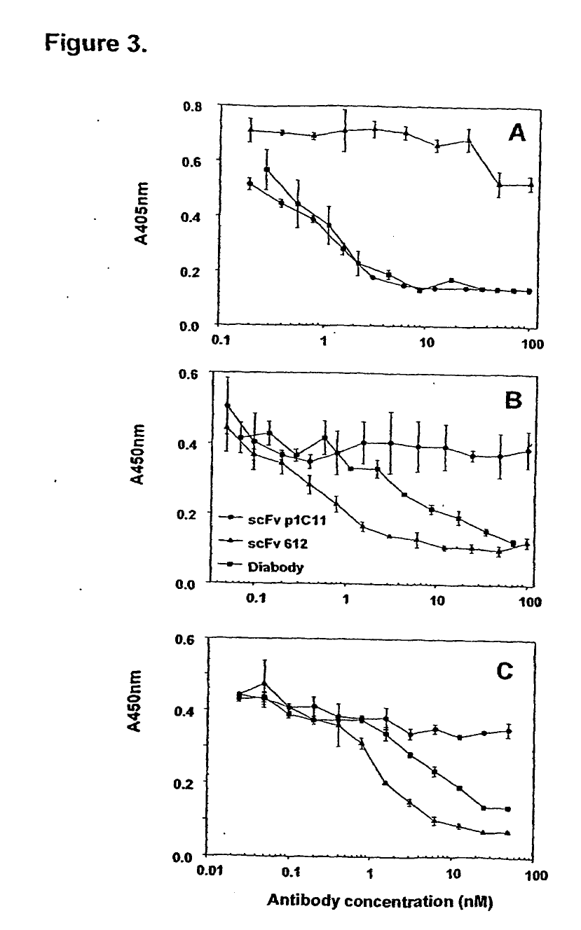

FIG. 3A shows that the diabody blocks KDR from binding to immobilized VEGF, in a dose-dependent manner as efficiently as scFv p1C11, with an IC₅₀ of approximately 2 nM. The diabody also blocks Flt-1 from binding to VEGF with an IC₅₀ of about 15 nM, which is about 10-fold less potent than the parent scFv 6.12 (FIG. 3B). Further, the diabody blocks PlGF, a Flt-1-specific ligand, from binding to immobilized Flt-1 with an IC₅₀ of approximately 4 nM (FIG. 3C). As expected, scFv p1C 11 had no effects on Flt1/VEGF and Flt-1/PlGF interaction, whereas scFv 6.12 had no effects on KDR/VEGF interaction. Data shown represent the mean ±SD of triplicate samples.

### EXAMPLE 3

### Biological Activity

### Inhibition of VEGF-Induced Migration of Leukemia Cells and Mitogenesis of HUVEC.

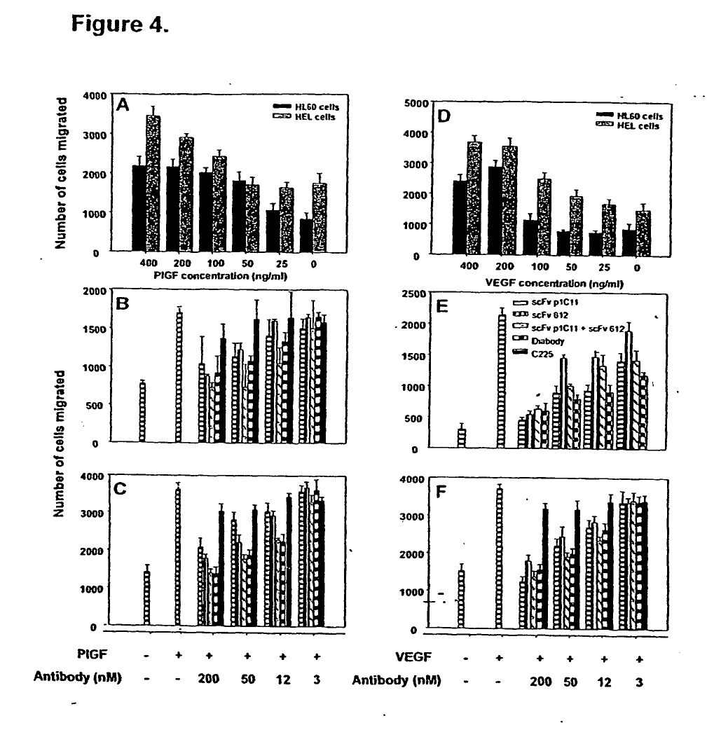

The diabody was first-tested for its activity in inhibiting VEGF and PlGF-induced cell migration. Both VEGF and PlGF induced migration of human leukemia cells, HL60 and HEL, in a dose-dependent manner (FIG. 4A and 4D). scFv p1C11 and scFv 6.12 effectively inhibited VEGF and PlGF-induced cell migration (FIG. 4B, 4C, 4E and 4F). Data shown are representative of at least three separate experiments and represent the mean ±SD of triplicate determinations. The two scFv showed a different efficacy pattern: scFv p1C11 is a stronger inhibitor of VEGF-induced cell migration, whereas scFv 6.12 is slightly more potent in inhibiting PlGF-induced cell migration. In contrast, the diabody is equally effective in blocking cell migration induced by both VEGF and PlGF. Combination of both scFv p1C11 and scFv 6.12, either as a simple mixture or in the diabody format, demonstrated a more potent inhibitory effect than either scFv alone. It is noteworthy that neither scFv p1C11 nor scFv 6.12 alone was able to completely inhibit VEGF or PlGF-induced cell migration, even at the highest antibody concentration tested (i.e., 200 nM). In contrast, combination of scFv p1C11 and scFv 6.12, either as a mixture or a diabody, completely abolished cell migration at an antibody concentration of 200 nM. A Fab fragment of C225, an antibody directed against epidermal growth factor receptor, did not show significant inhibition of cell migration in this assay.

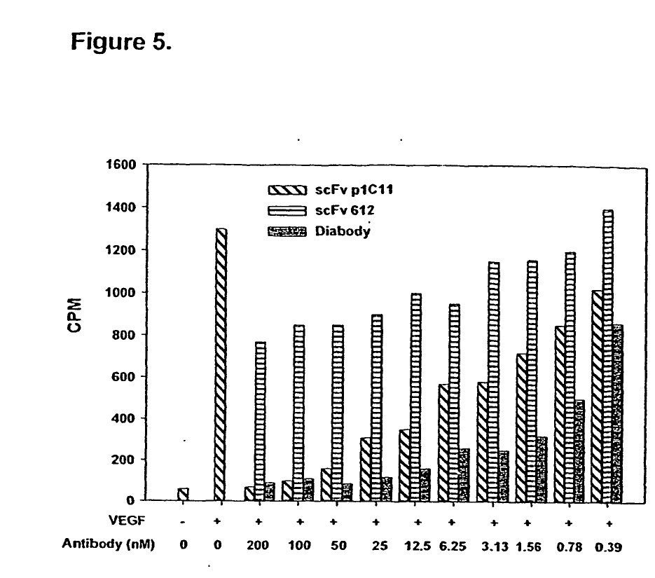

The VEGF-neutralizing activity of the bifunctional diabody was further determined using a HUVEC mitogenic assay. Data shown are the means of duplicates and are the representative of at least three separate experiments. As previously seen, scFv p1C11 effectively inhibited VEGF-stimulated HUVEC mitogenesis (measured by [³H]-TdR incorporation) in a dose-dependent manner with an IC₅₀ of approximately 2 nM. Anti-Flt-1 scFv 6.12 showed a very weak anti-mitogenic effect in this assay. The bifunctional diabody demonstrated a much stronger inhibitory effect than either scFv p1C11 and scFv 6.12 at every antibody concentration tested, with an IC₅₀ of approximately 0.5 nM (FIG. 5). Data shown are the means of duplicates and are the representative of at least three separate experiments.

What is claimed is:

1. An antibody having a first antigen binding site specific for a first VEGF receptor and a second antigen binding site specific for a second VEGF receptor.

2. The antibody of claim 1 wherein the first and second VEGF receptors are mammalian.

3. The antibody of claim 1 wherein the first and second VEGF receptors are human.

4. The antibody of claim 3 wherein the first and second VEGF receptors are selected from the group consisting of KDR, Flt-1 and Flt-4.

5. The antibody of claim 3 wherein the first VEGF receptor is KDR and the second VEGF receptor is Flt-1.

6. The antibody of claim 3 wherein the first VEGF receptor is KDR and the amino acid sequences of the complementarity determining regions (CDRs) of the first antigen binding site comprise:
SEQ ID NO: 1 at CDRH1;
SEQ ID NO: 2 at CDRH2;
SEQ ID NO: 3 at CDRH3;
SEQ ID NO: 4 at CDRL1;
SEQ ID NO: 5 at CDRL2; and
SEQ ID NO: 6 at CDRL3.

7. The antibody of claim 3 wherein the first VEGF receptor is KDR and the amino acid sequences of the variable domains of the first antigen binding site comprise:
SEQ ID NO: 7 for the heavy-chain variable domain (V_{H}); and
SEQ ID NO: 8 for the light-chain variable domain (V_{L}).

8. The antibody of claim 3 wherein the first VEGF receptor is KDR and the nucleotide sequences of the complementarity determining regions (CDRs) of the first antigen binding site comprise:
SEQ ID NO: 9 for CDRH1;
SEQ ID NO: 10 for CDRH2;
SEQ ID NO: 11 for CDRH3;
SEQ ID NO: 12 for CDRL1;
SEQ ID NO: 13 for CDRL2; and
SEQ ID NO: 14 for CDRL3.

9. The antibody of claim 3 wherein the first VEGF receptor is KDR and the nucleotide sequences of the variable domains of the first antigen binding site comprise:
SEQ ID NO: 15 for the heavy-chain variable domain (V_{H}); and
SEQ ID NO: 16 for the light-chain variable domain (V_{L}).

10. The antibody of claim 3 wherein the first VEGF receptor is KDR and the amino acid sequences of the complementarity determining regions (CDRs) of the first antigen binding site comprise:
SEQ ID NO: 1 for CDRH1;
SEQ ID NO: 21 for CDRH2;
SEQ ID NO: 3 for CDRH3;
SEQ ID NO: 4 for CDRL1;
SEQ ID NO: 5 for CDRL2; and
SEQ ID NO: 6 for CDRL3.

11. The antibody of claim 3 wherein the first VEGF receptor is KDR and the amino acid sequences of the variable domains of the first antigen binding site comprise:
SEQ ID NO: 22 for the heavy-chain variable domain (V_{H}); and
SEQ ID NO: 23 for the light-chain variable domain (V_{L}).

12. The antibody of claim 3 wherein the first VEGF receptor is KDR and the nucleotide sequences of the complementarity determining regions (CDRs) of the first antigen binding site comprise:
SEQ ID NO: 9 for CDRH1;
SEQ ID NO: 24 for CDRH2;
SEQ ID NO: 11 for CDRH3;
SEQ ID NO: 12 for CDRL1;
SEQ ID NO: 13 for CDRL2; and
SEQ ID NO: 14 for CDRL3.

13. The antibody of claim 3 wherein the first VEGF receptor is KDR and the nucleotide sequences of the variable domains of the first antigen binding site comprise:
SEQ ID NO: 25 for the heavy-chain variable domain (V_{H}); and
SEQ ID NO: 26 for the light-chain variable domain (V_{L}).

14. The antibody of claim 3 wherein the first VEGF receptor is KDR and the first antigen binding site comprises a set of amino acid sequences at CDRL1, CDRL2, CDRL3, CDRH1, CDRH2, and CDRH3, the set selected from the group consisting of the set of SEQ ID NOS:53, 54, 55, 65, 66, and 67, the set of SEQ ID NOS:56, 57, 58, 65, 66 and 67, the set of SEQ ID NOS:59, 60, 61, 65, 66, and 67, and the set of SEQ ID NOS:62, 63, 64, 68, 69 and 70.

15. The antibody of claim 3 wherein the first VEGF receptor is KDR and the first binding domain comprises a pair of V_{H} and V_{L} domains, the pairs selected from the group consisting of SEQ ID NOS:72 and 74, SEQ ID NOS:76 and 78, SEQ ID NOS:76 and 81, and SEQ ID NOS:83 and 85.

16. The antibody of claim 3 wherein the first VEGF receptor is KDR and the first antigen binding site comprises the set of amino acid sequences CDH1, CDRH2, and CDRH3 given by SEQ ID NOS: 65, 66, and 67, respectively, and a set of amino acid sequences at CDRL1, CDRL2, CDRL3 selected from the group consisting of the set of SEQ ID NOS:106, 107, and 108, the set of SEQ ID NOS:109, 110, and 111, the set of SEQ ID NOS:112, 113, and 114, the set of SEQ ID NOS:115, 116, and 117, the set of SEQ ID NOS: 18, 119, and 120, the set of SEQ ID NOS: 121, 122, and 123, the set of SEQ ID NOS: 124, 125, and 126, the set of SEQ ID NOS: 127, 128, and 129, the set of SEQ ID NOS: 130, 131, and 132, and the set of SEQ ID NOS:133, 134, and 135.

17. The antibody of claim 3 wherein the first VEGF receptor is KDR, the V_{H} domain of first binding domain comprises SEQ ID NO:76, and the V_{L} domain of the first binding domain comprises a sequence selected from the group consisting of SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO: 103, and SEQ ID NO:105.

18. The antibody of any one of claims 6 to 17 wherein the second VEGF receptor is Flt-1 and the second antigen binding site comprises the heavy chain and light chain variable domains of Mab 6.12 (ATCC No. PTA-3344).

19. The antibody of claim 3 wherein the first VEGF receptor is Flt-1 and the amino acid sequences of the complementarity determining regions (CDRs) of the first antigen binding site comprise:
SEQ ID NO: 35 at CDRH1;
SEQ ID NO: 36 at CDRH2;
SEQ ID NO: 37 at CDRH3;
SEQ ID NO: 38 at CDRL1;
SEQ ID NO: 39 at CDRL2; and
SEQ ID NO: 40 at CDRL3.

20. The antibody of claim 3 wherein the first VEGF receptor is Flt-1 and the amino acid sequences of the variable domains of the first antigen binding site comprise:
SEQ ID NO: 41 for the heavy-chain variable domain (V_{H}); and
SEQ ID NO: 42 for the light-chain variable domain (V_{L}).

21. The antibody of claim 3 wherein the first VEGF receptor is Flt-1 and the nucleotide sequences of the complementarity determining regions (CDRs) of the first antigen binding site comprise:
SEQ ID NO: 43 for CDRH1;
SEQ ID NO: 44 for CDRH2;
SEQ ID NO: 45 for CDRH3;
SEQ ID NO: 46 for CDRL1;
SEQ ID NO: 47 for CDRL2; and
SEQ ID NO: 48 for CDRL3.

22. The antibody of claim 3 wherein the first VEGF receptor is Flt-1 and the nucleotide sequences of the variable domains of the first antigen binding site comprise:
SEQ ID NO: 49 for the heavy-chain variable domain (V_{H}); and
SEQ ID NO: 50 for the light-chain variable domain (V_{L}).

23. The antibody of claim 3 wherein the first VEGF receptor is Flt-1 and the first antigen binding site comprises the heavy chain and light chain variable domains of Mab 6.12 (ATCC No. PTA-3344).

24. An antibody that binds specifically to an extracellular domain of a first VEGF receptor and an extracellular domain of a second VEGF receptor, wherein binding of the antibody to the first or the second VEGF receptor neutralizes activation of that VEGF receptor.

25. The antibody of claim 24 which blocks binding of VEGF.

26. The antibody of claim 24 which blocks receptor homodimerization.

27. The antibody of claim 24 which blocks receptor heterodimerization.

28. The antibody of claim 24 wherein the first and second VEGF receptors are selected from the group consisting of KDR, Fit-1 and Flt4.

29. The antibody of claim 24 wherein the first VEGR receptor is KDR and the second VEGR receptor is Flt-1.

30. An antibody that binds specifically to an extracellular domain of a first VEGF receptor and an extracellular domain of a second VEGF receptor and reduces tumor growth.

31. The antibody of claim 29 wherein the first and second VEGF receptors are selected from the group consisting of KDR, Flt-1 and Flt-4.

32. The antibody of claim 29 wherein the first VEGR receptor is KDR and the second VEGR receptor is Flt-1.

33. A antibody that binds specifically to an extracellular domain of a first VEGF receptor and an extracellular domain of a second VEGF receptor and inhibits angiogenesis.

34. The antibody of claim 32 wherein the first and second VEGF receptors are selected from the group consisting of KDR, Flt-1 and Flt-4.

35. The antibody of claim 32 wherein the first VEGR receptor is KDR and the second VEGR receptor is Flt-1.

36. A method for making an antibody having a first antigen binding site comprising a first immunoglobulin heavy chain variable domain and a first immunoglobulin light chain variable domain that specifically binds to an extracellular domain of a first VEGF receptor, and a second antigen binding site comprising a second immunoglobulin heavy chain variable domain and a second immunoglobulin light chain variable domain that specifically binds to an extracellular domain of a second VEGF receptor, which comprises
a) coexpressing in a host cell
a recombinant DNA construct encoding a first polypeptide having the first immunoglobulin heavy chain variable domain located to the N terminus of the second immunoglobulin light chain variable domain, and
a recombinant DNA construct encoding a second polypeptide having the second immunoglobulin heavy chain variable domain located to the N terminus of the first immunoglobulin light chain variable domain,
for a time and in a manner sufficient to allow expression of the polypeptides and formation of the antibody; and
b) recovering the antibody.

37. The method of claim 35 wherein the constructs are on the same DNA expression vector.

38. The method of claim 35 wherein the constructs are on different DNA expression vectors.

39. The method of claim 35 wherein the host cell is a bacterial cell, a yeast cell or a mammalian cell.

40. The method of claim 35 wherein the antibody is secreted from the host cell.

41. A method for neutralizing activation of a first VEGF receptor and a second VEGF receptor in a cell which comprises treating a cell with an antibody having a first antigen binding site specific for the first VEGF receptor and a second binding site specific for the second VEGF receptor in an amount sufficient to neutralize activation of the receptors.

42. A method for reducing tumor growth in a mammal in need thereof comprising treating the mammal with an antibody having a first antigen binding site specific for the first VEGF receptor and a second binding site specific for the second VEGF receptor in an amount effective to reduce tumor growth.

43. A method for inhibiting angiogenesis in a mammal in need thereof comprising treating the mammal with a bispecific antibody having a first antigen binding site specific for the first VEGF receptor and a second binding site specific for the second VEGF receptor in an amount effective to inhibit angiogenesis.
